(19) United States
(12) Patent Application Publication
(10) Pub. No.: US 2023/0087736 A1
(43) Pub. Date: Mar. 23, 2023

(54) BRAIN FUNCTION MAPPING WITH INTRACRANIAL ELECTROENCEPHALOGRAM (EEG) USING EVENT-RELATED SPECTRAL MODULATIONS

(71) Applicant: Children's Hospital Medical Center, Cincinnati, OH (US)

(72) Inventors: Ravindra Arya, Cincinnati, OH (US); Brian Ervin, Cincinnati, OH (US)

(21) Appl. No.: 17/798,190

(22) PCT Filed: Feb. 26, 2021
(86) PCT No.: PCT/US21/19995

§ 371 Date: Aug. 8, 2022

Related U.S. Application Data
(60) Provisional application No. 62/982,148, filed on Feb. 27, 2020.

(51) Int. Cl. A61B 5/37; A61B 5/31; A61B 5/291; A61B 5/374; A61B 5/00

(52) U.S. Cl. CPC A61B 5/37; A61B 5/31; A61B 5/291; A61B 5/374; A61B 5/4887

(57) ABSTRACT

A method for functional brain mapping using high gamma modulation obtained from stereoelectroencephalography (SEEG).

### CROSS REFERENCE TO RELATED APPLICATION

This application claims benefit under 35 U.S.C. § 119(e) of the U.S. provisional application No. 62/982,148 filed Feb. 27, 2020, the content of which is herein incorporated by reference in its entirety.

### FIELD OF THE INVENTION

The present disclosure relates to functional mapping of anatomical brain areas associated with brain activity, and more particularly, to a brain mapping method and system constructed using high gamma stereotactic electroencephalography (SEEG).

### BACKGROUND OF THE INVENTION

Precise preoperative assessment of the individual functional anatomy surrounding a brain defect or lesion is crucial for safe and effective neurosurgery. The risk of postoperative functional deficits are essential considerations when planning neurosurgical interventions. As each patient's brain anatomy is unique, brain mapping is not generalizable and must be done in a patient-specific manner.

Currently, intraoperative electrocortical stimulation (ECS) and intracarotid amobarbital test remain the clinical gold-standards for mapping brain functions and determination of dominant hemisphere respectively. These have typically used subdural grids. However, neurophysiologic and patient-safety concerns associated with these invasive procedures have fostered the development of alternative methods of functional brain mapping. For example, stereotactic electroencephalography (SEEG) has emerged as the preferred modality for intracranial monitoring in drug-resistant epilepsy (DRE) patients being evaluated for neurosurgery. After the implantation of SEEG electrodes, care is taken to determine the neuroanatomic locations of electrode contacts (ECs), to localize ictal (e.g., a seizure event) onset and propagation, and integrate functional information to facilitate surgical decisions. Typically, non-invasive neuroimaging modalities such as pre-operative magnetic resonance imaging (MRI) and post-operative EC implantation computed tomography (CT) scans are used in combination with SEEG to identify, sort, and label the SEEG ECs to generate a three-dimensional functional brain map. This process is often performed manually, which is resource intensive.

### SUMMARY OF THE INVENTION

The present disclosure is based, at least in part, on the development of methods for mapping functional areas of the brain with high specificity, accuracy, and sensitivity. For example, analysis of task-related high gamma modulations (HGM) using statistical thresholding of distribution of power differential clusters in time-frequency domain shows that SEEG HGM (e.g., in the 50-150 Hz range) signals were successfully used to adequately localize reference neuroanatomy and ESM (Electrical cortical Stimulation Mapping) speech/language sites with high accuracy, high specificity, and fair sensitivity across both hemispheres and grey matter (GM) as well as white matter (WM).

Accordingly, the present disclosure provides, in some aspects, a method for mapping a functional brain site in a subject. The method may comprise (a) subjecting a subject to a task; (b) performing an intracranial electroencephalography (iEEG) assay to the subject while the subject is performing the task, wherein the iEEG assay comprises multiple stereotactic or subdural electrodes placed at multiple sites within the subject's brain; (c) recording high gamma EEG signals during step (b) at each iEEG electrode; (d) analyzing the high gamma EEG signals to produce a collection of output data parameters; and (e) identifying a functional brain site in the subject that is responsible for performing the task based the collection of output data parameters determined in step (d). In some embodiments, stereotactic electrodes were used in step (b).

The subject may be a human patient such as a human child or a human adult. The subject may be in need of a functional brain map, for example, the subject has a brain disorder such epilepsy (e.g., drug-resistant epilepsy (DRE)), a brain tumor, or a vascular lesion, and may require surgical resection to remove the seizure focus, tumor or lesion. The method disclosed herein may be performed to the subject prior to a brain surgery. In one embodiment, the method further comprises determining a site for the brain surgery based on the functional brain site identified in step (e).

In any of the methods disclosed herein, the high gamma iEEG signals comprise signals at about 50-150 Hz.

In one embodiment, the analyzing step (d) comprises calculating time-frequency representations (TFRs) for frequency bands ranging from about 50-150 Hz, optionally with 1 Hz step. Alternatively or in addition, the analyzing step (d) comprises a clustering algorithm. In some examples, the clustering algorithm is a Maris-Oostenveld nonparametric permutation-based clustering procedure. In one example, the analyzing step (d) comprises locating the positions of electrode contacts by a manual procedure or a computer-assisted procedure such as FASCILE.

In some embodiments, the functional brain site is a language site and the task is visual naming, auditory naming, story listening, or conversational speech. In other embodiments, the functional brain site is a motor site and the task is visually-cued hand motor task.

Any of the methods disclosed herein may further comprises training a predictive model by comparing the analyses results obtained in step (d) with a reference database (e.g., the Neurosynth database).

Any of the methods disclosed herein may further comprises comparing the mapping results obtained in step (e) with a reference database (e.g., the Neurosynth database).

Any of the methods disclosed herein may further comprises using a predictive model to predict the probability that a SEEG electrode contact is located in a location in the subject's brain that participates in the task executed by the subject.

Alternatively or in addition, the method may further comprise performing ESM to the subject and comparing the mapping results obtained method with the results from the ESM.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

FIG. 1 includes diagrams showing high-gamma visual naming stereo-electroencephalograph (SEEG) maps projected on neuroanatomic language parcels. Axial, coronal, and left sagittal views of the SEEG electrodes for all patients. Neurosynth language parcels in the three views are shown as light gray shading. Upper panel: locations of electrode contacts that produced significant HGM during the performance of the visual naming task. Electrode contacts with significant HGM that co-localized within Neurosynth language parcels are considered as true positive and are displayed as small dark gray circles. Electrode contacts with significant HGM that are lying outside Neurosynth language parcels are considered false positive and are displayed as small light gray circles. Lower panel: the locations of electrode contacts (ECs) that did not produced significant HGM during the performance of the visual naming task. Electrode contacts without significant HGM that are lying outside Neurosynth language parcels are considered true negative and are displayed as small dark gray circles. Electrode contacts without significant HGM but co-localized within Neurosynth language parcels are considered as false negative and are displayed as light gray circles.

FIG. 2 includes diagrams showing significant predictors of high-gamma modulation (HGM) for localization of neuroanatomic language parcels. Kernel density distribution of time to largest power change cluster (T_{LZ}) showed a peak at 1.33 s that selected electrode contacts lying within Neurosynth language parcels (upper panel). T_{LZ} has been adjusted for individual patients according to their response times such that the start of naming after picture display for all patients is at 1 s. The frequency of the center-of-mass power change cluster (F_{CM}) showed peaks at 87 Hz and 103 Hz in the electrode contacts within Neurosynth language parcels (lower panel).

FIG. 3 includes diagrams showing visual naming associated HGM maps and ESM speech/language sites. Axial, coronal, and left sagittal views of all stimulated stereo electrode contacts for all patients. Upper panel: the locations of electrode contacts that produced significant HGM during the performance of the visual naming task. Electrode contacts with significant HGM and scored ESM+(true positive) are displayed as small dark gray circles, while electrode contacts with significant HGM but scored ESM−(false positive) are displayed as small light gray circles. Lower panel: the locations of electrode contacts that did not produced significant HGM during the performance of the visual naming task. Electrode contacts without significant HGM and scored ESM−(true negative) are displayed as small dark gray circles, while electrode contacts without significant HGM but scored ESM+(false negative) are displayed as small light gray circles.

FIG. 4 includes diagrams showing significant predictors of high-gamma modulation (HGM) for localization of ESM speech/language sites. Kernel density distribution of time to center-of-mass of the power change cluster with highest cluster weight (T_{CM}) showed a peak at 0.75 s that for selected ESM− sites (upper panel). T_{CM} has been adjusted for individual patients according to their response times such that the start of naming after picture display for all patients is at 1 s. The kernel density distribution of the value of power change of the largest cluster (V_{LZ}) showed two peaks at 1.47 and 1.83 log-units in ESM speech/language sites, compared to a single peak at 1.5 log-units in ESM− electrode contacts (lower panel).

FIG. 5 includes diagrams showing a probabilistic map of visual naming associated high-gamma modulation projected on neuroanatomic language parcels. Axial, coronal, and left sagittal views of the SEEG electrodes for all patients. Neurosynth language parcels in the three views are shown as light gray shading. Upper panel: the different gray and filled circles indicate the category of the EC: small dark gray circles are true positive (HGM model predicted EC to have language function and EC is within the Neurosynth language parcel), small light gray circles for false positive (HGM model predicted EC to have language function but EC is outside the Neurosynth language parcel). Outlines of the circles, if any, indicate the confidence of HGM model in prediction: dark outline=high model predicted probability of having language function. Please note that light gray circles with dark outlines (high model predicted probability but outside language parcels) are typically on the boundary of Neurosynth shading. Lower panel: the different gray and circles indicate the category of the EC: small dark gray circles are true negative (HGM model predicted EC not to have language function and EC is outside the Neurosynth language parcel), and small light gray circles are for false negative (HGM model predicted EC not to have language function but EC is within the Neurosynth language parcel). Outlines of the circles, if any, indicate the confidence of HGM model in prediction: dark outline=low model predicted probability of having language function. Please note that small light gray circles with dark outlines (low model predicted probability but inside language parcels) are typically on the boundary of Neurosynth shading.

FIG. 6 includes diagrams showing a probabilistic map of visual naming associated high-gamma modulation with respect to electrical stimulation speech/language sites. Axial, coronal, and left sagittal views of all stimulated stereo ECs for all patients. Upper panel: the different gray and filled circles indicates the category of the EC: small dark gray circles are true positive (HGM model predicted EC to have language function and ESM+), and small light gray circles are false positive (HGM model predicted EC to have language function but ESM−). Outlines of the circles, if any, indicate the confidence of HGM model in prediction, dark outline=high model predicted probability of having language function. Lower panel: the different gray and circles indicates the category of the EC: small dark gray circles are true negative (HGM model predicted EC not to have language function and ESM−), and small light gray circles are false negative (HGM model predicted EC not to have language function but ESM+). Outlines of the circles, if any, indicate the confidence of HGM model in prediction, dark outline=low model predicted probability of having language function.

### DETAILED DESCRIPTION OF THE INVENTION

Embodiments of the present disclosures are based, at least in part, on the unexpected development of methods involving intracranial stereo-electroencephalography signals (iSEEG) in association with event-related spectral modulations, which refers to the task-related variations of brain electrophysiological activity in the high gamma frequency range.

As a non-limiting example, high-gamma iSEEG was performed upon drug-resistant epilepsy (DRE) patients while having the patients perform a visual naming task. The high-gamma modulation (HGM) recorded in the iSEEG assay had non-parametric clusters of 50-150 Hz power modulations in the time-frequency domain. These high power modulation clusters were used to model and predict functional speech/language sites of the brain of these patients with high specificity and sensitivity. The method described here is a functional brain mapping method comprising HGM iSEEG. This mapping method was validated by two different and well known brain mapping standards, the reference neuroanatomy from Neurosynth using primarily the language sites, and electrical stimulation mapping (ESM) for the speech/language sites in the patients' brain.

Additionally, the present discloses are based, at least in part, the development of an alternative process to identify and label electrode contacts (ECs) of the implanted electrodes, a computer-assisted process that is both faster and user friendly for identifying and labeling ECs. Currently, the benchmark clinical way of identifying and labeling ECs is a manual method, typically performed by an experienced clinician. When compared to the benchmark clinical and manual method, this automated process reduces the average time it takes from 3.3 hours to 10.7 minutes, and the error rate is within an acceptable range. Moreover, the automated process can be performed by a first time clinician rather than an experienced clinician. When this automated process is performed in the high-gamma iSEEG functional mapping of brain method described herein, the mapping can be performed in a shorter period of time, thus reducing the time of the entire mapping procedure, the patient's exposure and risk of complication (e.g., seizures), operational discomfort, and overall cost. It also enhances accuracy, specificity, and sensitivity of the functional brain mapping.

### I. Functional Brain Site Mapping

Without being bound by theory, the method disclosed herein provides a predictive modelling incorporating time, frequency, and magnitude of power change and is a useful methodology for task-related HGM iSEEG, which offers insights into discrepancies between HGM brain functional maps (e.g., language maps) and neuroanatomy or ESM. The brain functional mapping obtained from any of the methods disclosed herein would have significant clinical importance, for example, to minimize the risk of damaging certain brain functional sites during neurosurgical treatment of DRE, brain tumors, or vascular lesions.

As a non-limiting example, the method described herein is useful for neurosurgeons to determine the functionally significant areas of the brain cortex within and adjacent to seizure-onset zone(s) of a subject having epilepsy. For example, when the method is applied for mapping language sites, such a method may comprise recording task-related HGM in an iSEEG assay, analyzing the task-related HGM using statistical thresholding of distribution of power differential clusters in time-frequency domain, and applying the output analysis in predictive modeling to map out the areas of the brain that are involved in language and/or speech. When the task is looking a picture of an object and verbally naming the recognized object immediately, the task-related HGM SEEG adequately localize to reference neuroanatomy Neurosynth language parcels, and classify accurately the ESM speech/language sites, the speech/language sites that were involved in the naming activity.

In some aspects, provided herein is a method for mapping a functional brain site in a subject, comprising: (a) subjecting a subject to a task; (b) performing an intracranial electroencephalography (iEEG) assay to the subject while the subject is performing the task, wherein the iEEG assay comprises multiple stereotactic or subdural electrodes placed at multiple sites on within the subject's brain; (c) recording high gamma EEG signals during step (b) at each stereotactic iEEG electrode; (d) analyzing the high gamma EEG signals; and (e) identifying a functional brain site in the subject that is responsible for performing the task based the collection of output data parameters determined in step (d). The preferred electrodes are the stereotactic or depth electrodes. A task is used in order to measure brain function or areas of the brain that are involved in executing that task. In some embodiments, the method further comprises utilization of FASCILE to identify and label the electrode contacts in the implanted electrodes.

Without being bound by theory, the present HGM mapping with iSEEG using stereotactic electrodes is superior to current clinical gold standard of (ESM) electrical stimulation mapping for the several reasons. ESM requires direct stimulation of the brain areas via the subdural electrodes. In contrast, the present mapping does not involve stimulation of the stereotactic electrodes but rather involves the HGM collected from the electrical activity in the brain when the brain is controlling the subject executing a task.

1) Electrical stimulation mapping is associated with risk of after-discharges (AD's), which are epileptiform discharges often occurring in runs, seen after termination of electrical stimulation. AD's threaten the neurophysiologic validity of mapping because it is uncertain if the observed behavioral response is due to electrical stimulation or AD's. Also, if the AD's are remote from the site of stimulation, then the neuroanatomic localization of observed behavioral response becomes unclear. HGM mapping is not associated with risk of AD's because it is a “passive” method and does not involve stimulation of the implanted electrodes.

2) Electrical stimulation mapping is associated with risk of unwanted seizures in some patients. This can be a challenging experience for the patient, family, and the clinical team. HGM mapping is not associated with any risk of unwanted iatrogenic seizures because it is a “passive” method and does not involve stimulation of the implanted electrodes.

3) Electrical stimulation mapping is typically performed in one or more sessions, each session lasting 1-3 hours. To compare, recording or data acquisition for HGM iSEEG mapping requires approximately 10-30 minutes. There is a six fold reduction of time a patient has to be subjected to a medical procedure. This is important because these patients are in the rather challenging environment of an epilepsy monitoring unit with implanted electrodes in their brain. Because of this reason, and possible pre-existing developmental or behavioral abnormalities, sustained patient cooperation may be challenging in some cases. Therefore, HGM iSEEG mapping is friendlier to the patient in these circumstances. The reduced medical procedure time also result in lower cost and resources (e.g., medical expertise, operation room etc.) usage. Thus, overall greater efficiency and effectiveness with comparable or better data without sacrificing specificity, accuracy, and sensitivity.

(i) Stereotactic Electroencephalogaphy (sEEG)

Stereotactic electroencephalogaphy (sEEG), also called stereo-EEG or sEEG, is an invasive procedure that utilizes localized, penetrating depth electrodes to measure electrophysiological brain activity. It is most commonly used in the identification of epileptogenic zones in cases of refractory epilepsy. Depth EEG electrodes (also called stereotactic electrodes) are those electrodes which are placed within the substance of the brain, for examples, into the white matter and the grey matter in the brain cortex, the temporal lobes, the amygdala and hippocampus. The electrodes are placed in targeted areas of brain. Typical, a sEEG procedure requires only small (2.5-mm) drill holes in the skull, unlike subdural grid implantation that requires a craniotomy, and the sEEG electrodes are stereotactically placed in the brain. SEEG electrodes typically have a diameter of 0.8 mm and can have up to 16 cylindrical platinum-iridium contacts, each 2 mm in length and spaced from 3 to 8 mm apart. These depth electrodes are placed through the tiny holes to explore large areas of the brain with minimal tissue damage.

Stereotactic as used herein relates to or denotes techniques for surgical treatment or scientific investigation that permit the accurate positioning of probes inside the brain or other parts of the body, based on three-dimensional diagrams. A stereotactic electrode as used herein refers to an electrode that has been accurately positioned inside the brain using three-dimensional diagrams or tools.

In general, a three-dimensional coordinate system is used to locate small targets inside the brain for implanting the sEEG electrodes. Specifically, the system can include plain X-ray images (radiographic mammography), computed tomography (CT), and magnetic resonance imaging (MRI) are used to guide the stereotactic placement of the electrodes. The depth of the electrodes and the stereotactic placement of the electrodes facilitate electrode contacts extending from the lateral cortical convexity to the medial cortex. Thus, implanted electrodes generally can provide a sampling of a unique set of brain regions including deeper brain structures such as hippocampus, amygdala and insula that cannot be captured by superficial measurement modalities such as electrocorticography (ECoG). Post-operative CT may be performed to verify the positions of the implanted sEEG electrodes. The placement of these electrodes may also be confirmed with co-registration on an MRI image.

Alternatively, electroencephalogram may be obtained using subdural electrodes. Subdural EEG electrodes are those electrodes which sit over the surface of the brain. To place these electrodes, the neurosurgeon performs a craniotomy where he makes a temporary window in the skull, expose the involved area of brain. The subdural electrodes are then lay down directly on the brain surface. The temporary bone window is replaced, and the incision is carefully closed. The placement of these electrodes are then confirmed with co-registration on an MRI san image or a CT image.

The stereotactic electrodes or the subdural electrodes can be placed in the right hemisphere, the left hemisphere, or in both hemispheres of the brain in the subject. Accordingly, the iEEG assay is performed in right hemisphere, the left hemisphere, or in both hemispheres of the brain in the subject.

Subsequent to the implantation of the electrodes, the positioned electrodes are then connected to a recording equipment that monitors and records the electrophysiological brain activity. The recordings or signals of a brain's electrical activity is called the brain electroencephalogram (EEG) and is recorded in the form of waveforms. These waveforms have two main characteristics: signal intensity and signal frequency. Signal intensity relates to height of the waveform and is measured in microvolts (mV). Signal frequency relates number of waveform per unit time and is measured in hertz (Hz). Spanning a frequency spectrum from below 3 Hz to over 30 Hz, there are five main frequencies of the human EEG waves. Delta: has a frequency of 3 Hz or below. It tends to be the highest in amplitude and the slowest waves. It is normal as the dominant rhythm in infants up to one year and in stages 3 and 4 of sleep. Theta: has a frequency of 4 to 7 Hz and is classified as “slow” activity. Alpha: has a frequency between 8 and 13 Hz. It is usually best seen in the posterior regions of the head on each side, being higher in amplitude on the dominant side. Beta: beta activity is “fast” activity. It has a frequency of 14-25 Hz, although this upper limit is variable in the literature. Gamma activity is the fastest brain wave, having a frequency above 25. The gamma spectrum of the EEG is further subdivided into low-gamma (typically 25-70 Hz), and high-gamma (above 50 Hz), although the terminology and exact frequency cut-offs are very variable in the literature. Gamma waveforms exhibit variations, rhythms and oscillations related to performance of a cognitive or motor task. The waveforms are collectively called modulations. These modulations are correlated with large scale brain network activity and cognitive phenomena such as working memory, attention, and perceptual grouping, and can be increased in amplitude via meditation or neurostimulation.

In some embodiments, the modulations in a brain's high gamma spectrum (e.g., of about 50-150 Hz) can be recorded and analyzed in order to formulate a predictive method of mapping the functional areas of the brain during cognitive activities such as speech, language, movement, and sensing. In some instances, the high gamma EEG signals comprise electrophysiological signals at about 50-150 Hz. A change or modulation in the 50-150 Hz range of power, separately for each 1 Hz bin, is calculated for the feedback response period compared to the pre-task resting state (baseline).

(ii) Subjects

The subject to be analyzed in the brain mapping method disclosed herein may be a human patient in need of having a functional brain mapped. In some instances, the human patient is a human adult (e.g., >18 yrs old or >21 yrs old). In other instances, the human patient is a human child (e.g., younger than 18, younger than 12, younger than 6).

In some embodiments, the subject has a brain disorder. Non-limiting examples of the brain disorder include epilepsy/seizures, a brain tumor, attention deficit/hyperactivity disorder (ADHD), Alzheimer's disease, a traumatic brain injury, a vascular lesion, a brain malformation and a learning disability. Examples of brain malformation may be congenital brain malformation such arteriovenous malformation (AVM) or vein of galen malformation. In one embodiment, the subject with drug-resistant epilepsy (DRE) where seizures sometimes are not controlled with anti-seizure medications. A number of different terms are used to describe DRE: “uncontrolled,” “intractable,” “refractory,” or “drug resistant.” About one-third of adults and approximately 20-25% of children have epilepsy that fails to come quickly under control with medicines in about. For these patients, resective epilepsy surgery is one of the few treatment available. Resective epilepsy surgery consists of removing the area of the brain that is causing the seizures. However, for a patient to be a good candidate for surgery, the area of the brain where seizures originate is clearly identified and the identified brain area of the brain can be safely removed with surgery. Therefore, the method described herein is especially useful for these DRE patients.

In some examples, the subject can be a human patient having a brain disorder and in need of a brain surgery. The brain mapping method disclosed herein may be performed prior to the brain surgery.

(iii) Tasks

In some embodiments, the task performed by the subject may be movement (motor function), tasting/feeling/sight/hearing/touch (sensory function), speaking (language function), and memorization (memory/recall function). For example, when the functional brain site is a language site and the task is visual naming—naming an object by sight; auditory naming—naming a sound by hearing, story listening—listen by hearing a story; or conversational speech—verbal speech in response to hearing on a specified conversational topic.

When the functional brain site is a motor site and the task is visually-cued hand motor task, that is movement produced in response to visual instructions. For example, a subject is cued to clicking a button. Such motor function assessment tasks are known in the art, e.g., in U.S. Pat. Nos. 9,028,256 and 9,653,002, the contents of which are incorporated by reference in their entirety.

When the functional brain site is a sensory site and the task can be having the subject identify a familiar object (e.g., coin, key) placed in the palm of the hand (stereognosis) and numbers written on the palm (graphesthesia) and to distinguish between 1 and 2 simultaneous, closely placed pinpricks on the fingertips (2-point discrimination). Such sensory function assessment tasks are known in the art, e.g., in International Patent Publication WO 2011/160222, and U.S. Pat. Nos. 7,610,096 and 9,619,613, the contents of which are incorporated by reference in their entirety.

When the functional brain site is a memory site and the task can be performing the word or picture learning and recall where the subject is allowed memorized five words or pictures, and after a few minutes is required to verbally recall the five words or pictures, verbal memory and visual memory respectively. Other memory related tasks include learning a story and recalling the story, learning a sentence memory followed by verbal recall of the sentence, learning a design memory and drawing the design. Other known memory assessment tool in the art include the Wide Range Assessment of Memory and Learning 2^{nd} edition (WRAML-2) and the U.S. Patent Publication No.: US 2017/0258383 and U.S. Pat. No. 9,420,970, the contents of which are incorporated by reference in their entirety.

(iv) Functional Brain Site Mapping

The described method can be used on a subject to map an important function brain site in the subject while the subject is performing a task. Brain mapping is a procedure that can help identify what different regions of the brain do. Different brain regions have specific functions. The exact location of certain functions (like movement, speech, vision, and more) differs quite a bit from person to person. This is particularly relevant for pediatric patients with neurological disorders, particularly DRE. A “map” of each person's brain can be made by stimulating certain brains areas. Alternatively, the person is asked to perform an activity and provide a feedback relating to functions such as movement, sensation, speech, vision, and the electrical activity in various brain areas is recorded during the course of the activity and feedback. The map tells the doctors just what parts of the brain are responsible for critical functions such as movement, sensation, or speech. Thus functional brain mapping allows for identifying the important areas of the brain, such as areas involved in motor, sensory, language, and memory functions. Functional brain mapping also allows for the correlation of abnormalities in various cortical networks with a patient's symptom's or deficits. The presence of tumors, seizures, or other brain abnormalities may change what parts of the brain control certain functions. With this knowledge, a neurosurgeon would have a “road” map to avoid compromising these important areas during neurosurgery. For example, the subject has a brain abnormality, disorder or disease. The brain abnormalities, disorder or disease may be causing some functional brain defects in the subject, e.g., a tumor growing in the visual cortex. Before performing any surgery on the brain, including epilepsy surgery, the surgeon wants to understand how the brain areas near the seizure onset or tumor function. This helps the neurosurgical team know how much of the seizure focus or tumor can be removed safely.

The term “mapping” or “map”, as used herein, in the context of functional brain mapping, refers to associating or linking an activity, symptom or deficit of the subject with one or more locations in the brain. The activity, symptom or deficit may be performed or experienced by the subject and is the functional aspect. For example, the activity is language recognition, language formulation or speech generation and the mapping is to associate the locations in the brain that control and may be involved in executing the language function in the subject. Alternatively, the activity is smelling (olfactory sensation) and the mapping is to link the locations in the brain that are involved in the intricate cognitive process of the smelling. Alternatively, the symptom is seizures and the mapping is to associate the locations in the brain that elicits the seizures. An example of a deficit is abnormal reflex and the mapping is to associate the locations in the brain that elicits the abnormal reflex.

The activity can be a cognitive activity. Cognitive activities are high-level activities such as problem solving, decision making, and sense making that involve using, working with, and thinking with information. These higher-level functions of the brain encompass language, imagination, perception, and planning.

The term “functional brain site” as used herein, in the context of functional brain mapping, refers to locations in the brain that control and/or are involved in allowing the subject to experience or performed an activity. The activity can be a cognitive activity. Thus, the functional brain site that can be mapped with the described method include but is not limited to movement site, sensory site, vision site, and memorization site.

When the subject performs a task as disclosed herein, the subject provide a consequential feedback, and the HGM is recorded. Preferably, there is contemporaneous time recording of the subject starting the task and providing the feedback, and the recorded brain activity is in the form of high gamma electrical activity of about 50-150 Hz produced during the performance of said task and feedback. Also preferably, the HGM brain activity is recorded from stereotactic electrodes in the subject's brain.

In analyzing the HGM signals recorded, the timing and extent of HGM power modulations may be considered, and a nonparametric statistical clustering algorithm may be used to find clusters of 50-150 Hz power modulations in the time-frequency domain, optionally with 1 Hz step. For example, the clustering algorithm can be Maris-Oostenveld nonparametric permutation-based clustering procedure. Clustering is a technology employed to simplify massive data by extracting essential information, based on which many subsequent analysis or processes become feasible or more efficient. Then a nonparametric distribution of the clusters of power difference between task feedback and resting state (baseline) in the 50-150 Hz frequency band, with 1 Hz bin is generated. Statistical analysis of the distribution of the clusters of power difference recorded is performed to determine those clusters that were statistically significant.

The analysis produces a collection of output data parameters, the collection of output data parameters are the time-frequency representations (TFRs). A time—frequency representation (TFR) is a view of a signal (taken to be a function of time) represented over both time and frequency. In one embodiment, the TFR may be calculated using Morlet wavelets. In the analysis, the collection of output data parameters produced (i) cluster weight (CW), (ii) probability of the largest time-frequency cluster arising from the baseline distribution (Pcluster), (iii) time of power change at the center-of-mass (T_{CM}), (iv) frequency of power change at the center-of-mass (F_{CM}), (v) value of power change at the center-of-mass (V_{CM}), (vi) time of the largest time-frequency differential (T_{LZ}), (vii) frequency of the largest time-frequency differential (F_{LZ}), and (viii) the value of the largest time-frequency differential (V_{LZ}). These output data parameter are input into a predictive model to predict the location in the brain that is involved in performing the task. Here, the predictive model is a generalized linear mixed model, and the output parameter used in the prediction model can be T_{CM}, F_{CM}, T_{LZ}, V_{LZ} or combinations thereof.

In one embodiment, the method further comprises identifying, group or sorting, and labeling the SEEG ECs within brain areas involved in the task that is performed by the subject. In one embodiment, the method comprises semi-automated localization and labeling of SEEG ECs for translation of HGM results into a spatial map in relevant brain spaces. The process of identifying, group/sorting, and labeling the ECs of functional significance can be performed using a software such as FASCILE (i.e., computer-assisted) or by any manual method that is known in the art. For example, a post-operative CT scan after the implantation of the stereo-electrodes can be used for identifying, group or sorting, and labeling the SEEG ECs. Any imaging modality that offers the visualization of both anatomical features and electrodes/contacts could be used.

For computer-assisted identification, sorting, and labeling the SEEG ECs, FASCILE may be used. FASCILE is a software for rapid and automated identification of ECs and it is an acronym for Fast Automated Stereo-EEG Electrode Contact Identification and Labeling Ensemble. The compiled Windows 64-bit executable is available at the website mega.nz/folder/lsRkwb5b #J6Ro_uD6X8kEXp1Vw-3UZA. The FASCILE software takes post-operative CT data, CTs that have been co-registered to MRI, and CTs that have been normalized to a standard brain space, and computes the locations of ECs from the input data, and allows the user to label the electrode arrays through a simple graphical user interface.

Image registration is the process of transforming different sets of data into one coordinate system. Image co-registration is performed when the intention is to study two or more images in a series, typically to understand change Images may come from the same or from different sensors, and have the same or different spatial resolutions. The rationale of co-registration is to ensure that the images become spatially aligned so that any feature in one image overlaps as well as possible its footprint in any other image in the series. For example, after the identification of the ECs from post-operative CT, these ECs are aligned with pre-surgical brain MRI, so that the location of EC's with respect to brain structures can be visualized.

The method further comprises determining a site for the brain surgery based on the functional brain site identified in step (e).

The described output parameters values obtained during a task (e.g., a language task or motor-button pushing task), such as T_{CM}, F_{CM}, V_{CM}, T_{LZ}, V_{LZ}, and F_{LZ} are input into a predictive model that outputs the probability of each EC having the associated-task function. This predictive model has been trained by comparisons with Neurosynth database. The predicted probabilities of EC's having an associated-task (e.g., language or motor) function is then displayed on the visualization obtained by co-registration of pre-surgical MRI and post-surgical CT using FASCILE or a manually labeled ECs.

The mapping results obtained for the method described herein may be compared with a reference database. This comparison may be used to train a predictive model. For example, the reference database Neurosynth's language parcels may be used to train a predictive model for mapping the language sites in the functional brain; the Neurosynth's sensory parcels may be used to train a predictive model for mapping the sensory sites in the functional brain; Neurosynth's motor parcels may be used to train a predictive model for mapping the motor sites in the functional brain and so forth. Non-limiting examples of reference databases are Neurosynth and BrainMap. These reference databases may be use used to train and or validate the predicted mapping results. Neurosynth is a platform for large-scale, automated meta-analyses of functional magnetic resonance imaging (fMRI) data. The platform and data are freely available at the website of Neurosynth organization. BrainMap is a platform and database of published functional and structural neuroimaging experiments with coordinate-based results (x,y,z) in Talairach or MNI (Montreal Neurological Institute) space. The platform and data are freely available at the website of BrainMap organization.

Additionally, an electrical stimulation mapping (ESM) can be performed to the subject and compared with the mapping results obtained the method described herein. When the functional brain site desired is a language site, a language ESM is performed. Similarly, when the functional brain site is a movement site, sensory site, vision site, or memorization site, a motor, sensory or memory/recall ESM respectively can be performed to the subject and the results can be compared to the mapping results obtained by the described method with the results from a respective ESM. ESM of the brain remains a major procedure for guiding epilepsy and tumor surgeries. Methods of brain ESM are known in the art, e.g., as described in the U.S. Pat. Nos. 8,738,140 and 8,874,220, the contents of which are incorporated by reference in their entirety. The ESM data are used to validate the predicted mapping results produced by the described method.

### II. Clinical Applications of the Brain Mapping Method

The method described herein may be performed to subjects prior to a brain surgery, preferably, in a subject in need of a functional brain map. For examples, the subject has a brain tumor and is in need of a brain tumor resection, the subject has epilepsy and is in need of a seizure focus resection, or the subject has a congenital malformation and is in need of stereotactic radiosurgery to treat AVM. The disclosed method provides vital information to the neurosurgeon to locate the area(s) for resection and area(s) to avoid, important areas of the brain that are involved in motor, sensory, memory, cognition, and language functions, and to determine whether the resection can be safely performed.

In addition, the method described herein can be used as a complement to ESM in pre-surgical evaluation of patients. Whereas ESM can require several hours, HGM mapping can be performed promptly in a few minutes. Therefore, in patients with limited ability to sustain cooperation (e.g., a young child), it may be worthwhile to first perform HGM mapping and then perform ESM only for those EC's that are HGM positive (+) and proximate to the seizure-onset zone. This is because, the described method using HGM has high specificity (ability to detect true negatives) compared to ESM. Both HGM iSEEG mapping (present disclosure) and ESM are performed in the same patient, using the same electrodes. The map produced by HGM iSEEG (present disclosure) in addition to ESM serves to provide a greater accuracy and a global picture of the studied functional brain sites for the neurosurgeon to make an informed medical decision. This is because while ESM only show sites that are critical to the tested function (test by electrical stimulation), HGM mapping shows all participating brain sites (i.e., areas involved and contributing to the execution of the task performed by the subject.

In some embodiments, the described HGM language mapping method may be particularly helpful in patients unable to undergo speech/language ESM. Therefore, in one embodiment, the subject is one who is unable to undergo speech/language ESM. For example due to delayed language development, or behavioral abnormalities precluding sustained cooperation with ESM.

Unless otherwise explained, all technical and scientific terms used herein have the same meaning as commonly understood by one of ordinary skill in the art to which this disclosure belongs. Definitions of common terms in neurobiology may be found in Fundamental Neuroscience for Basic and Clinical Applications edited by D. E. Haines and M. D. Ard (2013), published by Elsevier/Saunders; Brain Mapping: An Encyclopedic Reference edited by A. W. Toga, P. Bandettini, P. Thompson, and K. Friston, (2015), published by Elsevier; Pediatric Brain Stimulation: Mapping and Modulating the developing brain edited by A. Kirton and D. L. Gilbert (2016), published by Academic Press; and Clinical Brain Mapping by D. Yoshor and E.Mizrahi (2012), published by McGraw-Hill Education.

Unless otherwise stated, the present invention was performed using standard procedures known to one skilled in the art.

Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages will mean±1%.

All patents and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The present disclosure is further illustrated by the following example which should not be construed as limiting. The contents of all references cited throughout this application, as well as the figures and table are incorporated by reference herein.

Those skilled in the art will recognize, or be able to ascertain using not more than routine experimentation, many equivalents to the specific embodiments of the invention described herein. Such equivalents are intended to be encompassed by the following claims.

### EXAMPLE 1: High-Gamma Modulation Language Mapping with Stereo-EEG

Invasive pre-surgical evaluation of patients with is transforming due to a resurgence of stereo-electroencephalography (SEEG) fostered by advances in neuroimaging and robotics. This widespread adoption of SEEG reflects its relative safety and tolerability over subdural grids (5, 29). An important objective of invasive evaluation of DRE patients is to define the functional significance of cortex within and adjacent to seizure-onset zone(s). With subdural grids, electrical cortical stimulation mapping (ESM) is considered the gold-standard for such functional brain mapping (19). However, neurophysiologic and patient-safety concerns associated with ESM fostered development of an alternative methodology for functional mapping based on task-related power changes in EEG spectra, which has shown good accuracy for classification of ESM speech/language sites in multiple studies (4, 7, 31, 38, 40, 42, 45). Prior work on this high-gamma modulation (HGM) language mapping has been performed with subdural electrodes, with a paucity of studies on this modality with SEEG. Furthermore, analysis of HGM has been typically limited to computing the averaged power differential from baseline over the entire trial duration, to obtain binary classification of electrodes based on arbitrary statistical thresholds (4).

Described herein, is a method for analysis of HGM during a visual naming task, based on non-parametric distribution of clusters of power change in the time-frequency domain. This methodology was validated for language mapping with SEEG using predictive modeling, by comparing with functional neuroanatomic reference and ESM.

### Abbreviations

DOR Diagnostic Odds Ratio
FSIQ Full-scale Intelligence Quotient
GLMM Generalized Linear Mixed Model
PPV Positive Predictive Value
ROC Receiver Operating Characteristic
SD Standard Deviation

### Materials and Methods

### Participants and Signal Acquisition

All DRE patients undergoing SEEG monitoring at the Cincinnati Children's Hospital who were able to participate in the visual naming task were included, unless they had cortical malformation(s) or encephaloclastic lesion(s) involving the dominant perisylvian cortex. Hemispheric language dominance was decided according to the functional magnetic resonance imaging (MRI) if available, otherwise, left hemisphere was regarded as dominant unless the patient was left handed and had a left perisylvian cortical malformation (30). Stereotactic depth electrodes having 0.86 mm diameter and 2.41 mm contacts (Ad-Tech, Oak Creek, Wis.), were implanted in these patients for chronic monitoring. SEEG signals were sampled at 2048 Hz with a Natus Quantum amplifier using Neuroworks 8.5 software (Natus Neuro, Middleton Wis.). SEEG was recorded for HGM mapping using a referential montage, where one of the electrode contacts presumably farthest from the presumptive seizure-onset zone estimated on non-invasive pre-surgical evaluation, was empirically chosen as the reference. The study was approved by the institutional review board (#2017-4025). Informed consent from patients ≥18 years-of-age and parental permission in others, were obtained.

### Visual Naming

A series of 40 pictures were presented on a monitor using E-Prime 3.0 software (Psychology Software Tools, Sharpsburg Pa.). The pictures were displayed for 2-3 s (decided before testing, based on the patient's comfort) with is inter-stimulus interval. Patient was requested to name the picture aloud, immediately after the display. A trial run was performed before recording to eliminate pictures that the patient was unable to name, and the order of pictures was randomized before recording. Patient's audio output was collected with a microphone and routed through a digital trigger box (g.tec, Schiedlberg, Austria) to record the onset and termination of patient's voice on a separate channel synchronized to the SEEG data.

### Signal Analysis for SEEG HGM

Artefactual channels were rejected based on variance and average point-to-point difference being >5 standard deviations (SD) above mean. The remaining channels were notch filtered at 60 Hz and harmonics, and bandpass filtered at 50-150 Hz. The data were then divided into epochs aligned by termination of picture display, providing uniform 1 s pre-stimulus baseline. Epochs without a patient response or with excessively noisy baseline were excluded. A correction to remove low-frequency phase-locked responses was then applied to the epochs.

Time-frequency representations (TFRs) for 50-150 Hz frequency band, with 1 Hz step, were calculated using Morlet wavelets. For computation of TFRs, the number of cycles for the wavelet was f/5, for each frequency f, resulting in TFR for each epoch having 100 frequency bins. Since the distribution of baseline data in each frequency band was observed to be approximately log-normal, common logarithmic transformation was used. A change in 50-150 Hz power, separately for each 1 Hz bin, was then calculated for the response period compared to the pre-stimulus baseline. Statistical significance of TFRs was decided using Maris-Oostenveld nonparametric permutation-based clustering procedure (26). This required random sampling of baseline, with replacement across time and epoch, for each channel and frequency. The permutation-based clustering was performed with 2000 iterations, regular 2-dimensional lattice connectivity, time decimation factor of 8, and a frequency decimation factor of 2, resulting in 0.5 samples/Hz across 100 Hz bandwidth, and 85.3 samples/s across 3 s. These parameters were chosen to maximize the TFR resolution for the available computer memory.

For each electrode contact, all time-frequency points in each cluster were added to obtain cluster weight (CW). The largest cluster, having highest CW, was retained for further analysis. Time, frequency, and value of power change, at the center-of-mass of this cluster and at the point of largest time-frequency differential were noted (called T_{CM}, F_{CM}, V_{CM}, T_{LZ}, F_{LZ}, V_{LZ} respectively). The probability that the largest time-frequency cluster during patient response, arose from the baseline distribution (P_{CLUSTER}) was also calculated. These 8 metrics were input into predictive models described herein.

Onset of patient's verbal response was obtained from the microphone channel. To ignore filler words (e.g. “umm”), response onsets with z-score >±2 were rejected. The remaining responses were used to compute patient's mean response time. When combining across patients, T_{CM} and T_{LZ} were scaled to the individual mean response times. SEEG signal processing was implemented using Python 2.7 (MNE-Python 0.16.2) (17).

### Reference Neuroanatomy

To have a reproducible and generalizable reference to compare SEEG HGM naming maps, the Neurosynth database (see the website of neurosynth.org) was used, which performs a meta-analysis of published functional MRI studies based on specified criteria (43). The term “language” was used to search Neurosynth, and voxel-wise statistical inference maps were obtained from its automated parser. The language association test layer was then extracted as a 3D image dataset, from the Neurosynth output, as the reference neuroanatomy.

### Language ESM

A 50 Hz, bipolar, biphasic, square-wave stimulation was used, starting with 1 mA current, increased by 0.5-2 mA up to a maximum of 8 mA, until a functional response, evolving after-discharges (ADs), or a seizure occurred (3). The sequence of stimulated electrodes was individualized by the clinical team, based on proximity of the seizure-onset zone and the canonical language areas. An electrode from the deepest to the most superficial contact was stimulated, because of the concern that prior stimulation of cortex may possibly render the white matter contact(s) refractory to stimulation due to the preferential orthodromic conduction of stimulation impulse (41). Thus, each electrode contact was tested in 2 pairs, and was scored as having a language response (ESM+), only if speech/language interference was seen during both the stimulations. However, the contacts at the ends of the electrodes (deepest and the most superficial) could be tested in only one pair, and were scored according to the response on that single testing. Because speech/language errors during ESM can result from interference with cognitive and/or sensorimotor aspects of language function, and it is often not possible to separate the relative contributions of these components to the observed speech/language errors, any such interference was scored as ESM+, as per the usual clinical practice (3, 18). Contact pairs from different stereo electrodes were not stimulated in the ESM study.

### Image Processing

Pre-operative T1-weighted and Fluid Attenuation and Inversion Recovery MRI and post-operative computed tomographic (CT) scan (containing electrode locations) were co-registered with 6-parameter rigid-body deformation using the SPM12 toolbox in MATLAB. MRI images were first non-linearly warped into Montreal Neurological Institute (MNI) space using multi-channel segmentation. The same warping was then applied to the CT scan. Electrode contacts were detected from the warped CT scan, grouped by respective electrodes, and sorted from deep to superficial using an in-house algorithm. Electrodes were then labeled manually. After the electrode locations in MNI space were obtained, an intersection with Neurosynth reference anatomy was performed. This allows each electrode contact be labeled as lying within or outside a Neurosynth language parcel.

### Statistical Analysis

The patients were divided into 2 groups matched by the full-scale intelligence quotient (FSIQ), and randomly labeled as training and test subgroups. A mathematical model was developed in the training subgroup to predict the probability of electrode contacts lying within the Neurosynth language parcels, and the diagnostic performance of this model was evaluated in the test subgroup.

Input features to this predictive model included T_{CM}, F_{CM}, V_{CM}, T_{LZ}, F_{LZ}, V_{LZ}, CW, and P_{CLUSTER} A correlation matrix among these variables was calculated to exclude those with high internal correlation (Spearman's p>0.75). The following feature selection methods were then used to rank variables for inclusion in the predictive models: learning vector quantization, recursive feature selection with 10-fold cross-validated resampling, and Boruta algorithm. Variables which were consistently ranked best for localizing the Neurosynth language parcels, and were not significantly correlated, were input into generalized linear mixed models (GLMMs) with ‘patient’ as the random effect. The GLMMs were fitted using maximum likelihood estimation, and optimized using stepwise backward elimination. The most parsimonious model with minimum Akaike information criterion was retained. A nesting structure of electrodes within patients was pre-specified, since the electrodes cannot be regarded as independent observations. This model was completely independent of the test subset.

Best GLMM obtained from the training subset was then applied to the test subset, to obtain the probabilities of electrode contacts lying within the Neurosynth language parcels. Using receiver operating characteristic (ROC) to ascertain an optimal cut-off value, a binary probability was obtained for each electrode contact. Model performance was evaluated with area under the ROC curve (AUC). The model predicted location of the electrode contacts was compared with the Neurosynth locations to obtain diagnostic indices of SEEG HGM language mapping. Comparisons for subgroups of electrode contacts by the hemisphere (left/right), tissue (gray matter [GM]/white matter [WM]), and lobe (frontal/temporal) were performed. The relationships between the significant determinants and the contacts lying within the Neurosynth language parcels were explored to obtain insight into the analysis of SEEG HGM.

Finally, an identical analysis was performed to evaluate diagnostic performance of SEEG HGM compared to ESM speech/language sites as the reference standard. This analysis was limited to electrodes stimulated during language ESM. Statistical analysis was performed with R version 3.5 (34).

### Outcomes

The SEEG HGM naming maps, obtained using the approach described above, were compared with the Neurosynth anatomy. Electrode contacts were classified as true positive if HGM+ and lying within Neurosynth language parcels, false positive if HGM+but lying outside Neurosynth language parcels, true negative if HGM− and lying outside Neurosynth language parcels, and false negative if HGM− but lying within Neurosynth language parcels. Sensitivity, specificity, predictive values, likelihood ratios, and diagnostic odds ratio (DOR) along with their 95% confidence intervals (CI) were estimated. The hypothesis that DOR≠1 was tested with an omnibus Mantel-Haenszel test. A separate analysis was performed for comparisons between SEEG HGM and ESM language maps, including only the stimulated contacts.

### Results

Twenty-one patients (9 females), aged 4.8 to 21.2 years (mean±SD 12.9±4.5) were included. Of these, 18 patients were right-handed, 2 were left-handed, and 1 was ambidextrous. All patients were left hemisphere language dominant, according to the criteria described above (30). Their FSIQ ranged from 45-119 (79±19). The number of patients with right hemisphere, left hemisphere, and bilateral SEEG were 9, 7, and 5 respectively. From 7-20 electrodes (12±3) were implanted in each patient, having 72-266 (144±50) contacts. The total number of electrode contacts was 3022 (Table 1).

TABLE 1
Demographic and Clinical Information for the Included Patients

| | Age | | | | | | | Interlctol |
|---|---|---|---|---|---|---|---|---|
| ID | (y) | Sex | Hand | Onset (y) | Etiology | FSIQ | Semiology | EEG |
| 1 | 21.2 | m | R | 4 | Peri-ventricular | 51 | R | RT |
| | | | | | heterotopia | | | |
| 2 | 9.8 | f | R | 8 | Sequel of encephalitis | 45 | L | LF, LT, LO |
| 3 | 16.3 | f | R | 3 | MRI negative | 61 | X | B |
| 4 | 10.3 | m | R | 9 | MRI negative | 64 | T, F | RT |
| 5 | 20.8 | f | L | 15 | MRI negative | 92 | Cingulate | B |
| 6 | 16.4 | f | R | 16 | Mesial Temporal | 75 | RT, Insula | RT |
| | | | | | Sclerosis | | | |
| 7 | 11.4 | m | R | 7 | MRI negative | 68 | F | Normal |
| 8 | 12.6 | m | R | 0.1 | Encephalomalacia | 73 | RT | R > L |
| 9 | 19.1 | m | A | 6 | MRI negative | 112 | RO, LO | RTO |
| 10 | 7.4 | m | R | 4 | Mesial Temporal | 119 | T | LC |
| | | | | | Sclerosis | | | |
| 11 | 16.9 | m | R | 11 | Sequel of | 71 | X | RP, LOT |
| | | | | | meningoencephalitis | | | |
| 12 | 11.6 | m | R | 7 | MRI negative | 92 | F | LF, LT |
| 13 | 15.3 | f | R | 4 | Cortical Malformation | 78 | RF, RP, Insula | RCP, LCP |
| 14 | 8.9 | f | L | Neonatal | Encephalomalacia of | 76 | RF | RFT, RO |
| | | | | | Medial Right Thalamus | | | |
| 15 | 11.3 | m | R | 8 | Cortical Malformation | 95 | LF | LF, LT |
| 16 | 10.2 | m | R | 6 | DEPDC5 mutation, | 86 | F | LP |
| | | | | | Cortical dysplasia | | | |
| 17 | 14.6 | f | R | 1 | Mesial Temporal | 103 | T | LT |
| | | | | | Sclerosis | | | |
| 18 | 6.5 | m | R | 3 | Sequel of neonatal IVH | 71 | X | RT > LT |
| 19 | 14.9 | f | R | 10 | CNS Vasculitis | 75 | LF | L > R |
| 20 | 4.8 | f | R | Neonatal | Sequel of neonatal IVH | 90 | R | RC, RP, RT |
| 21 | 10.5 | m | R | 5 | MRI negative | 72 | F | BF |

| | Ictal | | | SPECT/ | | | Implanted |
|---|---|---|---|---|---|---|---|
| ID | EEG | MEG | PET/SPM | SISCOM | Electrodes | Contacts | Hemisphere |
| 1 | RT | RTO | RT | RF | 7 | 92 | R |
| 2 | MF | LP, LT | LTO | LH | 19 | 244 | B |
| 3 | LF, LT | Normal | RF | LF, LT | 16 | 188 | B |
| 4 | RT | RT, Insula | RT | Not done | 11 | 138 | R |
| 5 | LF | LP | X | Not done | 9 | 126 | L |
| 6 | RT | RT | RT | RT | 9 | 98 | R |
| 7 | Midline | RF | RF, RT, RP | RT | 16 | 212 | B |
| 8 | RT | RF, RP | RP | Not done | 10 | 96 | R |
| 9 | RTO > LTO | RO, RT, Insula | X | RF | 20 | 266 | B |
| 10 | LT | LP, Insula | LT | Not done | 11 | 118 | L |
| 11 | RP | X | RF | RO | 13 | 122 | R |
| 12 | LFT | LT | LT | LT, RF | 13 | 148 | L |
| 13 | RCP | RP, LP | R | R | 13 | 154 | R |
| 14 | RFT | RF, RT | RP | X | 12 | 162 | R |
| 15 | LF, LT, RF | LT, LF, RP | LT | LT | 12 | 150 | L |
| 16 | X | LF | X | RF, RP, L | 7 | 72 | L |
| | | | | Insula | | | |
| 17 | T | LT | Not done | LT | 9 | 106 | L |
| 18 | RT | RP, RT | R | RF, RT | 12 | 156 | R |
| 19 | X | LF, LT > RP | L | Not done | 11 | 120 | L |
| 20 | R | RO, RP | Not done | RT, RP | 12 | 100 | R |
| 21 | BF | Not done | LT, LP | LF, LT | 13 | 154 | B |

Comparison with Neurosynth Reference Anatomy

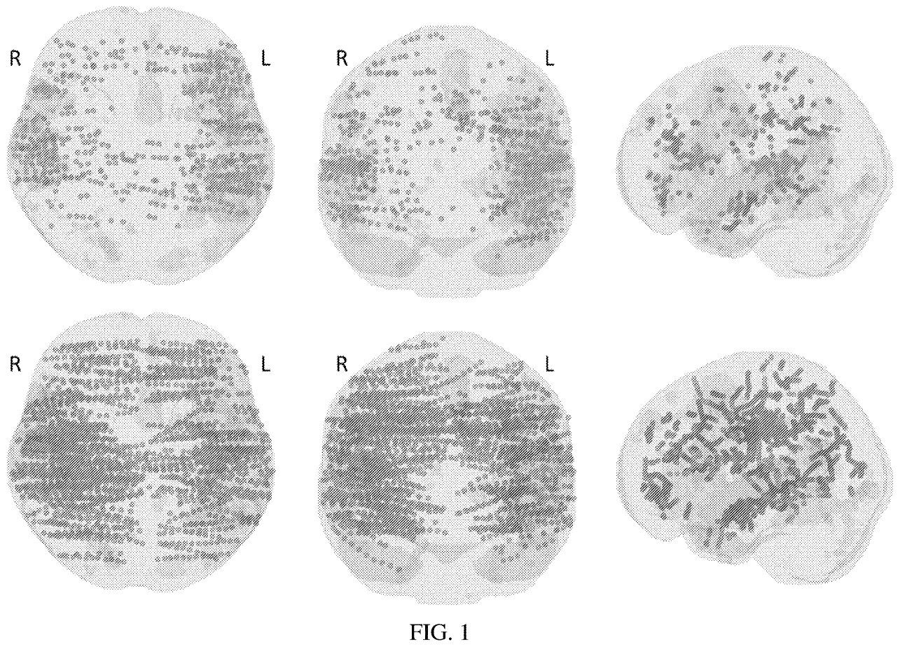

The GLMM for SEEG HGM developed in the training subset predicted the locations of electrode contacts within Neurosynth language parcels in the test subset with a high DOR (10.91, 95% CI 8.06, 14.78, p<0.0001, Table 2). SEEG HGM was highly accurate (0.81, 95% CI 0.78, 0.83), highly specific (0.85, 95% CI 0.84, 0.86), and fairly sensitive (0.66, 95% CI 0.61, 0.70). SEEG HGM showed a significant DOR for classification of Neurosynth language parcels in the left hemisphere (10.44), right hemisphere (11.67), GM (9.84), WM (11.91), frontal lobe (11.50), and temporal lobe (9.34, FIG. 1).

TABLE 2
Comparison of SEEG HGM probabilistic maps with neuroanatomic language parcels

| | | Left | Right | Gray | White | Frontal | Temporal |
|---|---|---|---|---|---|---|---|
| | All | hemisphere | hemisphere | Matter | Matter | lobe | lobe |
| Number of | 1314 | 613 | 701 | 673 | 621 | 319 | 384 |
| electrodes | | | | | | | |
| Accuracy | 0.81 (0.78, | 0.78 (0.75, | 0.83 (0.80, | 0.80 (0.77, | 0.81 (0.78, | 0.80 (0.75, | 0.75 (0.71, |
| | 0.83) | 0.81) | 0.85) | 0.83) | 0.84) | 0.84) | 0.79) |
| Sensitivity | 0.66 (0.61, | 0.65 (0.60, | 0.67 (0.58, | 0.63 (0.57, | 0.69 (0.62, | 0.72 (0.62, | 0.65 (0.59, |
| | 0.70) | 0.70) | 0.75) | 0.69) | 0.75) | 0.81) | 0.69) |
| Specificity | 0.85 (0.84, | 0.85 (0.82, | 0.85 (0.84, | 0.85 (0.83, | 0.84 (0.82, | 0.82 (0.79, | 0.84 (0.80, |
| | 0.86) | 0.87) | 0.87) | 0.87) | 0.86) | 0.84) | 0.87) |
| Positive | 0.57 (0.53, | 0.70 (0.64, | 0.41 (0.35, | 0.58 (0.52, | 0.56 (0.51, | 0.53 (0.46, | 0.76 (0.69, |
| Predictive | 0.61) | 0.74) | 0.46) | 0.64) | 0.62) | 0.60) | 0.81) |
| Value | | | | | | | |
| Negative | 0.89 (0.88, | 0.82 (0.80, | 0.94 (0.93, | 0.88 (0.86, | 0.90 (0.88, | 0.91 (0.88, | 0.75 (0.71, |
| Predictive | 0.90) | 0.84) | 0.96) | 0.90) | 0.92) | 0.94) | 0.78) |
| Value | | | | | | | |
| Likelihood | 4.39 (3.74, | 4.28 (3.36, | 4.52 (3.54, | 4.27 (3.37, | 4.40 (3.50, | 3.96 (2.92, | 3.96 (2.89, |
| Ratio | 5.12) | 5.45) | 5.57) | 5.36) | 5.44) | 5.14) | 5.49) |
| Positive | | | | | | | |
| Likelihood | 0.40 (0.35, | 0.41 (0.35, | 0.39 (0.29, | 0.43 (0.36, | 0.37 (0.29, | 0.34 (0.23, | 0.42 (0.35, |
| Ratio | 0.46) | 0.49) | 0.51) | 0.52) | 0.46) | 0.49) | 0.51) |
| Negative | | | | | | | |
| Diagnostic | 10.91 | 10.44 (6.92, | 11.67 (7.00, | 9.84 (6.47, | 11.91 | 11.50 | 9.34 (5.63, |
| Odds Ratio | (8.06, | 15.77) | 19.54) | 15.00) | (7.55, | (6.00, | 15.57) |
| | 14.78) | | | | 18.82) | 22.22) | |
| Mantel- | p < 0.0001 | p < 0.0001 | p < 0.0001 | p < 0.0001 | p < 0.0001 | p < 0.0001 | p < 0.0001 |
| Haenszel | | | | | | | |
| Test | | | | | | | |

[HGM High-Gamma Modulation; SEEG Stereo-electroencephalography]

### SEEG HGM Determinants of Neurosynth Language Parcels

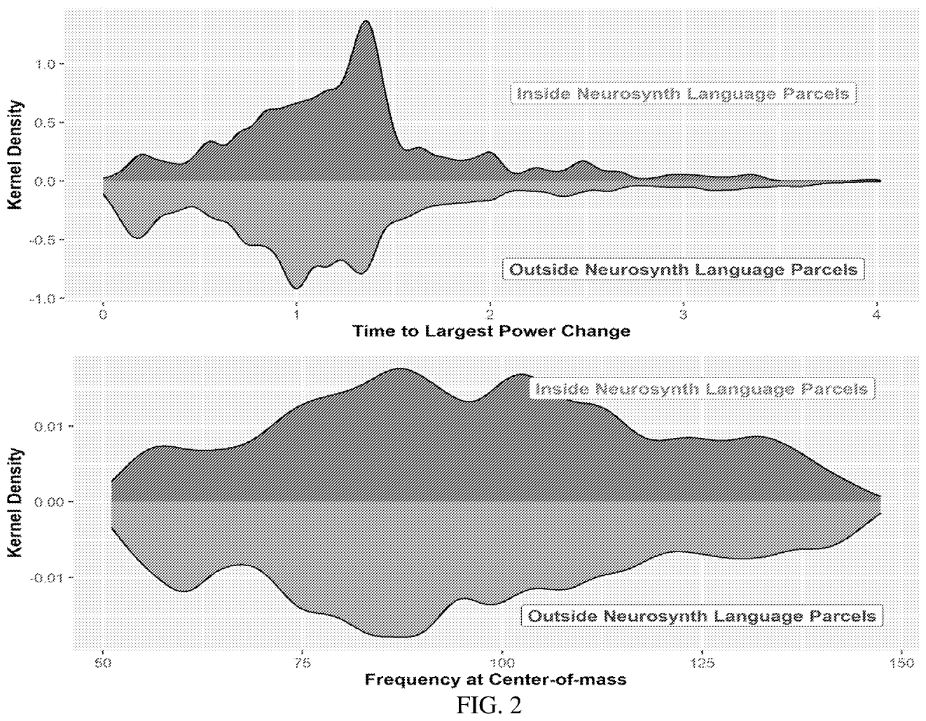

T_{LZ} and F_{CM} were found to be significant determinants of the localization of electrode contacts within Neurosynth language parcels (Table 3). The kernel density distribution of T_{LZ} showed a single peak at 1.33 s in the electrode contacts lying within Neurosynth language parcels, compared to a multimodal distribution in those outside language parcels (FIG. 2). Similarly, the F_{CM} distribution showed 2 peaks at 87 Hz and 103 Hz in the electrode contacts within Neurosynth language parcels, compared to a relatively flatter distribution in electrodes outside language parcels (FIG. 2).

TABLE 3
Optimal generalized linear mixed model for prediction of neuroanatomic
language parcels with SEEG HGM

| Random effects | Variance | SD | |
|---|---|---|---|
| Patient | 0.14 | 0.38 | |
| Fixed effects | Estimate | SE | p-value |
| T_{LZ} (scaled) | 0.19 | 0.07 | 0.0080 |
| F_{CM} (scaled) | 0.22 | 0.07 | 0.0032 |

Model Fit:
Area under the receiver operating characteristic curve 0.703

Comparison with ESM Speech/Language Sites

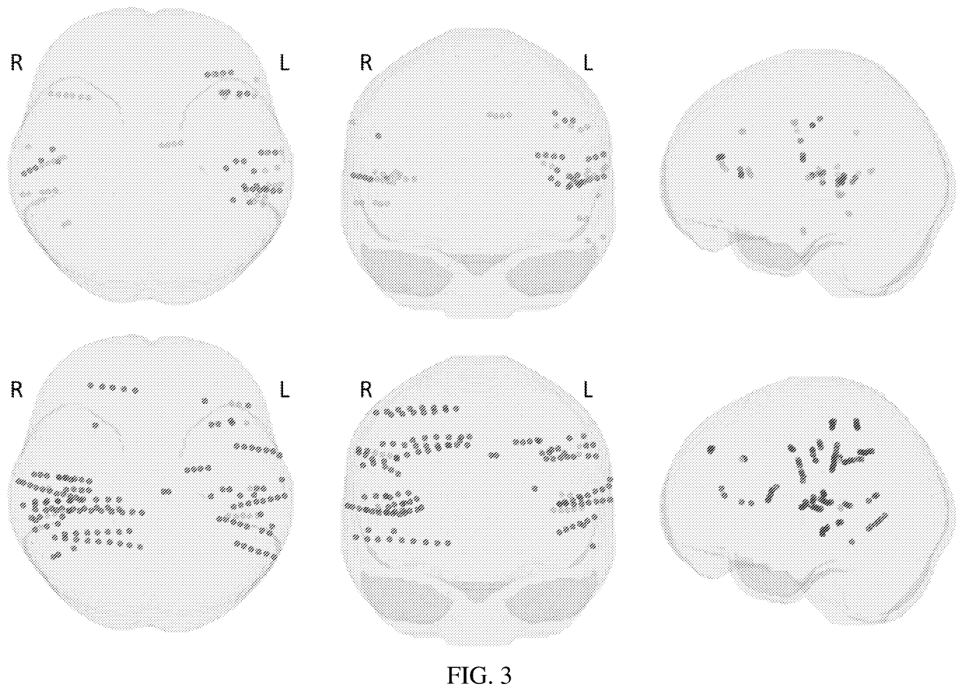

Using a separate model developed in the training subset, SEEG HGM provided a significant classification of ESM speech/language sites in the test subset (DOR 5.02, 95% CI 1.78, 14.45, p<0.0001, Table 4). SEEG HGM was found to have high specificity (0.74, 95% CI 0.69, 0.78) and negative predictive value (0.88, 95% CI 0.81, 0.93) but insufficient sensitivity and positive predictive value. Similar diagnostic performance was seen in GM, WM, temporal lobe, and frontal lobe, although there were fewer data points available for frontal lobe comparisons (FIG. 3). SEEG HGM showed excellent diagnostic performance in the left hemisphere (DOR 9.25, p<0.0001) with good sensitivity (0.78, 95% CI 0.56, 0.92) and specificity (0.73, 95% CI 0.65, 0.78) (Table 4).

TABLE 4
Comparison of SEEG HGM probabilistic maps with electrical stimulation mapping
(ESM) speech/language sites

| | | Left | Right | Gray | White | Frontal | Temporal |
|---|---|---|---|---|---|---|---|
| | All | hemisphere | hemisphere | Matter | Matter | lobe | lobe |
| Number of | 113 | 69 | 44 | 51 | 62 | 11 | 56 |
| electrodes | | | | | | | |
| Accuracy | 0.72 (0.63, | 0.74 (0.62, | 0.68 (0.61, | 0.67 | 0.77 | 0.91 | 0.71 (0.60, |
| | 0.79) | 0.81) | 0.80) | (0.54, | (0.65, | (0.57, | 0.77) |
| | | | | 0.73) | 0.87) | 0.91) | |
| Sensitivity | 0.64 (0.45, | 0.78 (0.56, | 0.29 (0.05, | 0.80 | 0.53 | 1.00 | 0.80 (0.47, |
| | 0.80)* | 0.92) | 0.66)* | (0.47, | (0.30, | (0.37, | 0.96)* |
| | | | | 0.96)* | 0.73)* | 1.00)* | |
| Specificity | 0.74 (0.69, | 0.73 (0.65, | 0.76 (0.71, | 0.63 | 0.84 | 0.88 | 0.70 (0.62, |
| | 0.78) | 0.78) | 0.83) | (0.55, | (0.77, | (0.64, | 0.73) |
| | | | | 0.67) | 0.91) | 0.88)* | |
| Positive | 0.41 (0.29, | 0.50 (0.36, | 0.18 (0.03, | 0.35 | 0.53 | 0.75 | 0.36 (0.21, |
| Predictive | 0.51)* | 0.59) | 0.42) | (0.21, | (0.30, | (0.28, | 0.44) |
| Value | | | | 0.42) | 0.73)* | 0.75)* | |
| Negative | 0.88 (0.81, | 0.90 (0.80, | 0.85 (0.80, | 0.93 | 0.84 | 1.00 | 0.94 (0.84, |
| Predictive | 0.93) | 0.97) | 0.93) | (0.81, | (0.77, | (0.73, | 0.99) |
| Value | | | | 0.99) | 0.91) | 1.00) | |
| Likelihood | 2.45 (1.43, | 2.83 (1.57, | 1.18(0.18, | 2.19 | 3.43 | 8.00 | 2.63 (1.25, |
| Ratio Positive | 3.70) | 4.12) | 3.87)* | (1.06, | (1.31, | (1.02, | 3.59) |
| | | | | 2.96) | 8.25) | 8.00) | |
| Likelihood | 0.49 (0.26, | 0.31 (0.10, | 0.94 (0.41, | 0.32 | 0.55 | 0.00 | 0.29 (0.05, |
| Ratio | 0.80) | 0.69) | 1.33)* | (0.05, | (0.29, | (0.00, | 0.85) |
| Negative | | | | 0.95) | 0.91) | 0.99) | |
| Diagnostic | 5.02 (1.78, | 9.25 (2.28, | 1.24 (0.14, | 6.93 | 6.20 | N/A | 9.14 (1.48, |
| Odds Ratio | 14.45) | 40.75) | 9.55)* | (1.12, | (1.44, | | 72.27) |
| | | | | 54.93) | 28.17) | | |
| Mantel- | p < 0.0001 | p < 0.0001 | p = 0.814 | p = 0.014 | p = 0.004 | p = 0.01 | p = 0.004 |
| Haenszel Test | | | | | | | |

*crosses point of no effect which is 0.5 for accuracy, sensitivity, specificity, and pretictive values; and 1 for likelihood ratios and diagnostic odds ratio.
[HGM High-Gamma Modulation; SEEG Stereo-electroencephalography]

### SEEG HGM Determinants of ESM Speech/Language Sites

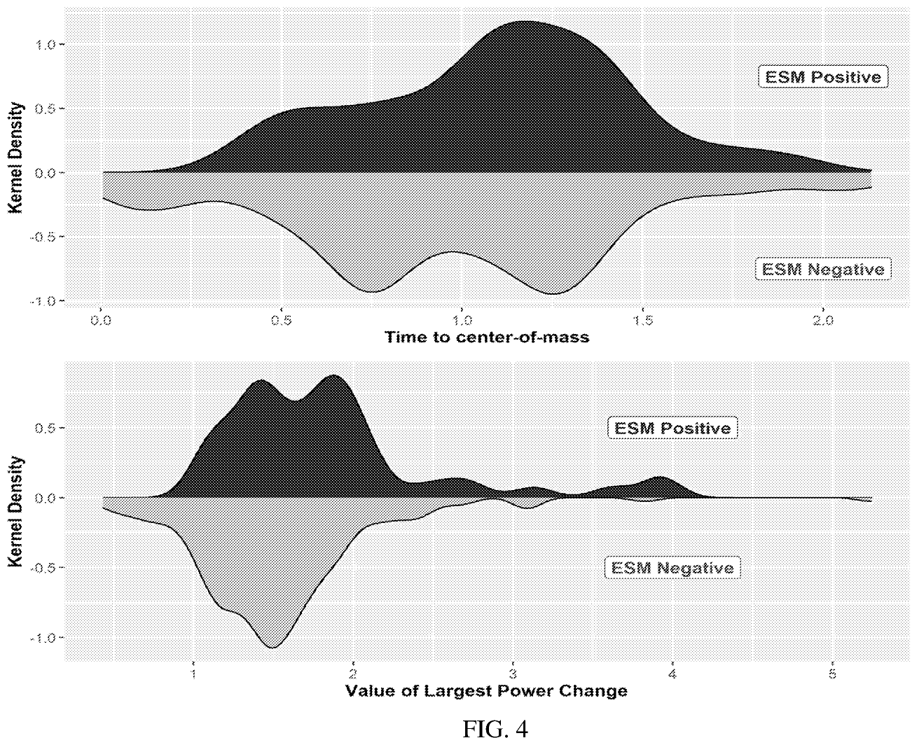

T_{CM} and V_{LZ} were found to be significant determinants of ESM+ sites. The kernel density distribution of T_{CM} showed a single large peak at 1.2 s in ESM+ electrode contacts, compared to bimodal peaks (1.25 s and 0.75 s) in ESM− sites (FIG. 4). ESM+ sites showed a bimodal distribution of the V_{LZ} peaking at 1.47 and 1.83 log-units, compared to a single peak at 1.5 log-units for ESM− sites.

The methodology described herein used non-parametric distribution of clusters of power differential between response and baseline and predictive modelling. The power differentials were HGM derived from intracranial SEEG. The method showed that SEEG HGM localized to language neuroanatomy with high accuracy, high specificity, and fair sensitivity across both hemispheres and GM as well as WM. SEEG HGM can also localize ESM speech/language sites with high specificity, particularly in the left hemisphere. This example provided insights into the analysis of HGM and found that it may be better to consider the timing and extent of power modulations, rather than averaging the power differential over the entire trial.

Clinical Implications for Language Mapping with SEEG

Given the high specificity and negative predictive value of SEEG HGM for localization of ESM speech/language sites, perhaps the most important clinical application of SEEG HGM is in pre-selection of electrodes for ESM. Conventional ESM is time and resource intensive, strenuous on the patient and clinical team, and is associated with risks of ADs and seizures, which compromise patient safety and neurophysiologic validity of ESM (9). Furthermore, the traditional anatomy based selection of electrode contacts for stimulation has been shown to have a poor positive predictive value (PPV) of 0.22 (3). To compare, the PPV of HGM model was nearly twice that at 0.41 (Table 4). Hence, HGM-based targeting for ESM may prove more useful in patients with aberrant or reorganized language networks, as HGM is agnostic to anatomical location.

A recent study has reported 92.4% specificity and 8.9% sensitivity for SEEG HGM compared to ESM in adults (14). Comparatively, this study found slightly lower specificity (74%), but much better sensitivity (64%) and more localized HGM responses. In time-frequency space, it is common to see multiple clusters of power difference between task and resting state in any given frequency band (26). This method generated a distribution of these clusters and performed statistical thresholding, resulting in more local HGM responses.

In an exemplary clinical application of the method, a HGM iSEEG is first performed because it is time-efficient and does not pose a risk of iatrogenic seizure(s). After this HGM iSEEG mapping, then an ESM can be performed to verify electrode contacts which are HGM+ and lie within/adjacent to seizure-onset zone. In patients where adequate ESM cannot be performed due to frequent ADs or induced seizure(s), the HGM iSEEG mapping model offers a probabilistic ranking of the likelihood of electrode contacts being ESM+, which may be harnessed for surgical decisions.

Functional Mapping with Subdural Grids Vs. SEEG

SEEG is often criticized for lacking the cortical surface contiguity afforded by subdural grids for mapping the boundaries of functional cortices. In this practice, functional significance of electrode contacts lying within a gyrus was ascertained, and surgical resections were decided based on the anatomy of the sulci delimiting that gyrus. Hence, mapping with SEEG whether using HGM or ESM, is not inferior to subdural grids, rather the surface contiguity of grids is replaced by the ability to sample from the deeper structures with SEEG.

Moreover, while the spread of electrical current down the pyramidal cell assemblies, on stimulation of subdural discs, is fairly well understood (22, 35), there is paucity of data on biophysics of SEEG ESM (20). With subdural ESM, there is some uniformity of stimulation of apical dendrites of pyramidal cells, acting as a defined input to the downstream neuronal system. However, stimulation of a stereo electrode contact which traverses multiple tissue types, may result in variable activation of different cell types. Hence, there is insufficient basis to assume that the interpretation of behavioral responses seen with SEEG ESM in clinical settings, are analogous to subdural ESM (3, 44).

Studies comparing HGM and ESM language maps in patients with subdural electrodes have shown widely varying sensitivity (0.23-0.99) and specificity (0.48-0.96) (11, 12, 27, 28). A meta-analysis of 15 studies found pooled sensitivity of 0.61 and specificity of 0.79, with significant heterogeneity in language tasks, techniques, and data analysis (4). These results (Table 4), further attested comparability of language mapping with SEEG and subdural grids. In addition, the HGM-model showed consistent localization of Neurosynth language parcels in both hemispheres, GM and WM, and frontal and temporal lobes (Table 2). SEEG HGM also localized ESM speech/language sites in frontal and temporal lobes, GM and WM, but only in the left hemisphere (Table 4). The interpretation of referentially recorded HGM is more straightforward compared to ESM across pairs of SEEG electrode contacts which may lie in different tissue types (GM and WM).

Insights into Discrepancies Between HGM and Neurosynth Anatomy or ESM

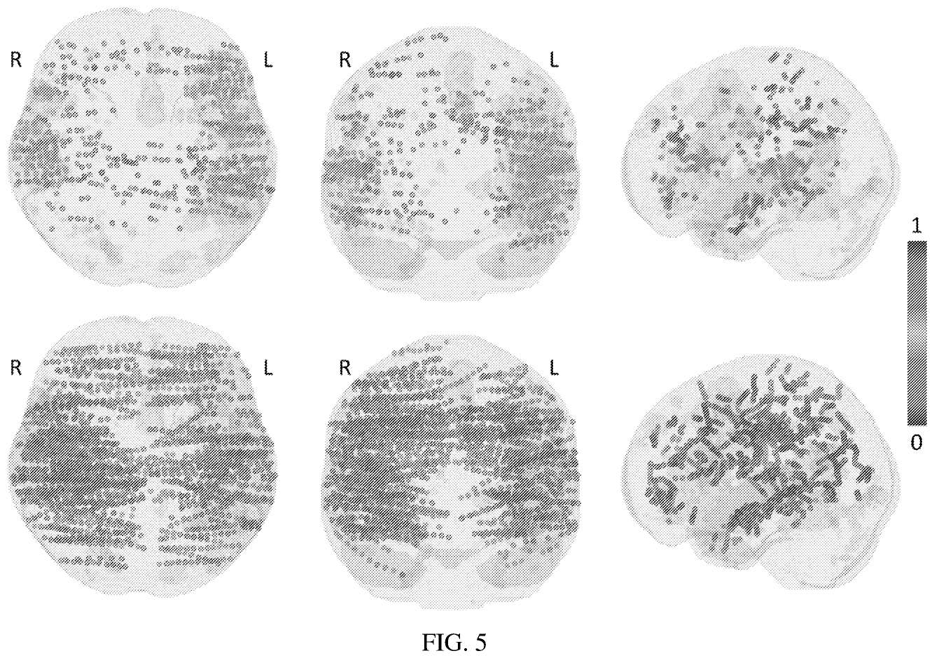

This study also showed that it may be more useful to analyze the timing, frequency, and magnitude of power modulations during a cognitive task, rather than averaging over the entire trial (12, 21, 36, 40). This helped understand the differences between HGM and Neurosynth language maps. The kernel density distribution of T_{LZ} identified significant clusters around 0.2, 1.0, and 1.33 times the response time (FIG. 2). However, only the peak occurring at 1.33 response time positively identified Neurosynth language parcels. This peak probably corresponded to auditory error checking and self-modulation after speech (3, 13, 32). Whereas, the peaks at 0.2 and 1.0 response time most likely corresponded to initial visual processing and oral motor function respectively, which was excluded from the Neurosynth map based only on meta-analysis of fMRI studies of language (33, 43). This was further supported by FIG. 5, showing groups of electrode contacts having high probability of language function, in left temporal, left frontal, and right temporal lobes, corresponding to bilateral auditory cortices and left perisylvian language areas. However, in the sagittal view, a number of high-probability electrode contacts were seen in inferior precentral gyrus, corresponding to oral motor area, which was excluded from the Neurosynth map.

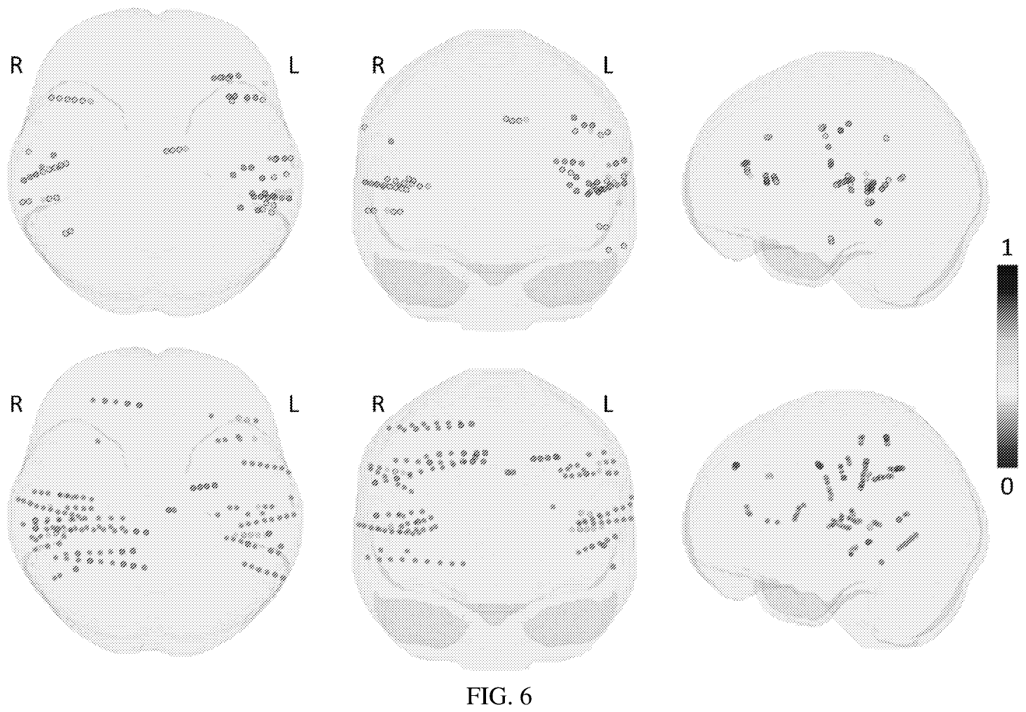

Similarly, the T_{CM} peak at 1.2× response time indicates the ESM+ electrodes (FIG. 4). However, given the peak at 1.25× response time indicates ESM− electrode contacts, the best differentiator for ESM+/− was the peak at 0.75× response time, which most likely reflects verbal working memory (word retrieval after object recognition). Consistent with earlier studies, the clusters with higher high-gamma power change, best identified ESM+ sites (see FIGS. 4 and 6). While a kernel density peak around 1.5 log-units was seen in both ESM+ and ESM−, a second peak at 1.83 log-units differentiated ESM+contacts from the ESM− contacts.

The peaks observed at 0.15, 1.2 and 1.25× in T_{CM} for ESM model were comparable to those at 0.2 and 1.33× in T_{LZ} for HGM model, probably representing similar physiology (FIGS. 2 and 4). Since T_{LZ} and T_{CM} were significantly correlated (p=0.99), both of these variables were not included in any one of the predictive model described herein. Instead, only one of these two variables is selected to be included in the model for analysis and prediction. The feature selection method favored T_{LZ} for HGM model and T_{CM} for ESM model. These peaks in kernel densities may reflect components of the visual naming task captured by HGM, which can potentially be used to model the underlying cognitive process. The observation that HGM representing working memory process negatively selected for ESM+ electrode contacts, led to speculation that ESM did not identify brain regions participating in verbal working memory. This hypothesis was supported by a series of patients with subdural grids, where resection of ESM−HGM+ electrodes was associated with significant working memory deficits (3). Hence, it is possible that this methodology for analysis of HGM can be utilized to study spatiotemporal processing of components involved in a cognitive task, which is not feasible with ESM.

### EXAMPLE 2: Fast Automated Stereo-EEG Electrode Contact Identification and Labeling Ensemble

Drug-resistant epilepsy (DRE) patients often require intracranial electroencephalography (EEG) to accurately define seizure-onset zone and functional areas (46). Two modalities in clinical use for chronic intracranial EEG include subdural electrodes implanted through a craniotomy, and stereotactic depth electrodes implanted through burr holes, called stereo-EEG (SEEG). In the last decade, SEEG has emerged as the preferred modality for intracranial monitoring, as shown by a 1.5-times increase in its utilization between 2000 and 2016 (47). In large meta-analyses, SEEG has also been shown to be safer and better tolerated than subdural grids (5, 29).

An important challenge with intracranial EEG is to localize electrode contacts (ECs) in relation to the neuroanatomy. With subdural grids, intraoperative photographs facilitate localization of ECs with sufficient accuracy, and are often used for identifying locations of ECs with ictal onset or functional significance for surgical planning. However, ascertaining precise anatomic locations of SEEG ECs remains an issue, because intraoperative photographs cannot be obtained for SEEG, and because it is a 3D modality. Although there are tools for co-registration of pre-operative magnetic resonance imaging (MRI) and post-operative computed tomography (CT) (48, 49); the sorting and labeling of SEEG ECs is often performed manually, which is labor intensive. Also, some of the software for semi-automatic segmentation and labeling of EC's may not be feasible for centers with limited resources and expertise.

To address these challenges, a software called Fast Automated SEEG Electrode Contact Identification and Labeling Ensemble (FASCILE) was developed and validated with convenient user interface and capabilities. FASCILE is not primarily meant for co-registration of CT and MRI as there are commercial software available for this purpose. Rather, it is designed to expedite localization of ECs, assigning ECs to respective SEEG electrodes (sorting), and labeling them.

### Methods

### Participants

All DRE patients undergoing SEEG evaluation were eligible for inclusion. Informed consent from patients ≥18-years-of-age, and parental permissions in others, were obtained. The study was approved by the institutional review board (#2017-4025).

### Imaging and SEEG Hardware

MRI—Pre-operative 3.0T-MRIs were performed using Philips Ingenia (Best, The Netherlands) or GE Signa Architect (GE Healthcare, Waukesha, Wis.) scanners. The protocol included: sagittal isotropic 1 mm 3D T1-weighted gradient recalled echo (1 mm axial and coronal multi-planar reformations), axial FSE T2-weighted (3-4 mm slice thickness at 3-4.5 mm intervals), axial T2-FLAIR (4 mm slice thickness at 4.5 mm intervals), coronal oblique T2-FLAIR (perpendicular to the plane of hippocampus, 3-4 mm slice thickness at 3-4 mm intervals), and sagittal isotropic 1 mm 3D T2-FLAIR (1 mm axial and coronal multi-planar reformations) sequences (50).

CT—Post-implant CT scans were performed on the Canon Aquilon One scanner (Canon Medical Systems) with age specific technique (mA 250, kVp100, 0.5 mm slice thickness acquisition, 1 mm slice thickness reconstructions, no scan angle).

SEEG—SEEG electrodes used for monitoring had 0.86 mm diameter and 2.41 mm long platinum contacts (AdTech, Oak Creek, Wis.).

### Identification, Sorting, and Labeling of Electrode Contacts

Clinical Method—At the center, identification, sorting, and labeling of SEEG ECs was performed manually using the Ortho Sections Viewer in Analyze 7.0 (48). The resolution of CT and MRI was modified to 0.8³ mm³. An intensity threshold was set manually, leaving only high intensity voxels. Teeth and wires were selected and removed from the image. Each electrode was selected manually and labeled using the surgical plan as a reference. Once all electrodes were labeled, ECs were identified within the electrodes. Starting with the deepest EC and moving superficially, crosshairs were placed on the visually-determined center of the EC, and an image of the fused CT-MRI was captured to generate a display slide, showing the three orthogonal views at the selected coordinates. Coordinates were not saved, and no overlay electrode NIfTI file could be created. This image processing was always performed by a single person (LR).

Automated Identification, Sorting, and Labeling of Electrode Contacts with FASCILE-FASCILE employed a novel automated method for identifying, sorting, and labeling ECs. FASCILE was written in Python 3.8.3, and used the following modules: Numpy 1.19.1, SciPy 1.5.0, Scikit-image 0.16.2, MNE 0.20.7, and NiBabel 3.1.1. Visualization module, also written in Python, used Matplotlib 3.3.1, Python-pptx 0.6.18, and Tkinter (17, 48, 51-58). FASCILE can be used with post-operative CTs, CTs co-registered to MRI using a variety of methods, and CTs normalized to a standard brain space. Described herein is the development and functionality of the FASCILE software.

Launcher—When first activated, FASCILE application launcher required the user to set a path for post-operative CT data in the NIfTI format. In addition, the user was able to set paths for directories containing MRI data in NIfTI format, SEEG as an EDF file, and input patient information (name, date of birth, and medical record number). MRI and EDF files were not essential for the function of FASCILE but allowed user-friendly enhancements. EDF file, which contains EC names, facilitated faster and easier labelling, while MRI data allowed super-imposing EC locations onto MRI sections. The “Begin” button submitted the user input and started the localization and labeling process. The “Exit” button terminated the program.

Preprocessing—CT data was read by the NiBabel module, and thresholded such that values below 3071 HU were set to 0 based on the evidence that typical CT bone window range lies between 226 to 3071 HU (50). Metal ECs were reliably outside this range. This threshold could be manually changed in case of non-conventional CT images, for example, cone-beam CT. CT data was then reshaped so that each dimension shared the same resolution using SciPy's zoom function.

Localization—Coordinates for potential locations of ECs were found in two steps. First, all local maxima in the thresholded CT data from the pre-processing step were found, using Scikit-image's peak_local_max function. The objective was to find all ECs, even at the cost of identifying some additional high HU artifacts. Secondly, groups of connected voxels were found using 7-point orthogonal connectivity with Scikit-image measure.label function. These groups were expected to represent some physical object (e.g. one or more ECs, a wire, or a set screw). Each group was thresholded at its mean, and the weighted centroids were found for the resulting objects. These centroids were added to the list of local maxima from the first step to give all the potential EC locations. More than 85% of the EC locations found using local maxima coincided with the locations found using weighted centroids when viewed from the axial, coronal, and sagittal views (data not shown).

If the image was saturated (all values above an upper limit were set to that upper limit), neither the local maxima approach nor the weighted centroid approach would perform adequately. This occurred with Philips cone-beam CT, which scales intensity units to roughly reflect HUs, but saturates at 3000. In this case, a Euclidean distance transform was applied (SciPy's distance_transform_edt function) to a binary thresholded image, then the local maxima approach was repeated. Locations found using all three approaches were then used in the grouping process.

Grouping—It was crucial to correctly group ECs lying on respective SEEG electrodes. In FASCILE, grouping was performed based on pairwise distance of potential ECs. Before any grouping was done, an expected distance was established based on the knowledge of the SEEG electrodes depending on the manufacturer, the image resolution, and the distribution of pairwise distances based on the SEEG implantation plan for an individual patient. Once the dimensions of the ECs and the spacing between them was obtained, the expected distances were translated from the real-world measurements to the pixel measurements, the data now having the same resolution in all dimensions (see above). The insulated spacing between the ECs was treated as the minimum possible distance, but the expected distance range was calculated based on the distribution of pairwise distances.

With the range of expected distances known, certain computational assumptions were made about ECs belonging to the same electrode. The challenge was to correctly group ECs that may have common neighbors belonging to different electrodes. Therefore, FASCILE used the proposition that an electrode contact EC_{A} belonged to a given electrode if and only if there was at least one other contact EC_{B} in the group within the expected distance range of EC_{A}. This was essentially similar to a graph where ECs are nodes, edges between these nodes indicated that ECs were within the expected distance range, and the electrodes were the constituent subgraphs.

Next, to automatically assign the ECs to their respective electrodes, FASCILE's grouping algorithm started by fitting a vector to subgroups of ≥3 ECs within each group. The quality of fit and the number of ECs fit by the vector were weighted to identify the vector (and associated ECs) that best described a given group. This subgroup of ECs was referred to as the group's “fit-set” and addressed the issue of multiple EC subgroups/vectors within each group. The fit-set was then extended by projecting to coordinates where an EC was expected to be, and assigning the EC nearest to the projected coordinates to the same set (electrode), if a nearby EC existed within the group defined by expected distance. The fit-set was then sorted from deep to superficial, based on the distance from the center coordinate of the CT data. Once this process was completed for each group, the expected distance range was revised based on the distribution of the distances between neighbors for all fit-sets. Finally, all ECs included in the fit-set were removed from consideration, and the grouping process was repeated until no new fit-sets are found.

Labeling—The nomenclature of SEEG is variable across institutions and precludes fully automatic labelling. Hence, FASCILE had a graphical user interface (GUI) which aided the user to label the electrodes quickly and accurately. The GUI presented four display panes: sagittal view, coronal view, axial view, and 3D scatter plot which could be rotated by the user.

During labelling, the fit-set under consideration was shown in red. All ECs in fit-sets yet to be considered were shown in blue, and all ECs in fit-sets which were already labeled were shown in green. In the three directional panes, left clicking added an EC near the mouse coordinates to the current fit-set. A right click removed the nearest EC from the fit set.

The user could specify the label and the number of ECs in an electrode using the entry boxes in the GUI. Alternatively, if the user had specified an EDF file containing electrode names in the launcher, a column of label buttons appeared in the GUI. Clicking a label button automatically defined the number of ECs on the electrode from the number of channels with the selected label in the EDF file. If the user wished to override the EC count, the count entry box in the GUI took precedence over the ECs automatically counted from the EDF file. The electrode label buttons automatically advanced to the next fit-set. Therefore, user needed to ensure that the correct number of ECs were entered before pressing the label button.

If the user made an error, the Restart button discarded all labels and other changes made up to that point, and restarted the labelling process from the beginning. The Reset button reloaded the fit-set currently under consideration. The Next button advanced to the next fit-set, and submitted user input (label and/or count). The Finish button skipped all remaining fit-sets, accepting only what had already been submitted.

Often, fit-sets include coordinates found within set screws due to their high HU values. FASCILE allowed the user to remove these ECs by right clicking, but this was not mandatory. The EC count (specified by user input or EDF file) cut off the extra high HU artifactual points, and the numbers 1 to EC count were assigned to ECs belonging to an electrode from deep to superficial. Occasionally, other high-HU objects, such as wires or teeth were also included in the fit-sets. When the current fit-set did not correspond to an electrode, the user could press the Next button without specifying a label or count. If there were multiple such artifactual fit-sets, the user could iterate through them, or directly press the Finish button, and FASCILE automatically removed these artifactual fit sets.

If an electrode was inadvertently missed or excluded during this process, the Add button allowed the user to manually add a new fit-set. To add a fit-set manually, the user first clicked on the deepest EC in any two of the three anatomical views, so that its coordinates in all three dimensions were specified. The same process was then repeated for the second deepest EC. Once these two ECs were added to the new fit-set, the user was returned to the labeling interface, where the new fit-set appeared in blue and was labeled in the end.

Output—The output from FASCILE consisted of three objects: a spreadsheet (.csv), a NIfTI electrode overlay file (.nii), and a Microsoft PowerPoint slide deck. The spreadsheet contained names of ECs, and their x (lateral), y (antero-posterior), and z (supero-inferior) coordinates. The spreadsheet was automatically named “LabelLocations<CT filename>.csv”, thereby avoiding ambiguity in case FASCILE was used on more than one CT. The NIfTI overlay file had the same dimensions and affine as the user-selected CT, but with all zeros beside the coordinates of the ECs, which were assigned the value of one. This facilitated visualization of ECs when overlaid on patient's CT or MRL

In addition, the user could choose to obtain a .csv file containing neuroanatomic parcel assignments for all ECs. These parcel names were assigned to each EC by intersecting its location in the Montreal Neurological Institute (MNI) space with standard neuroimaging atlases (MICCAI, Harvard-Oxford, AAL, and AAL2 atlases). However, this output would only be selected when the input CT file had been normalized to the MNI space.

### Comparative Analyses

FASCILE was validated against the clinical process. For each patient, ECs with the same label were matched, distances in x (lateral), y (antero-posterior), and z (supero-inferior) directions were obtained, and mean absolute distances in the 3 directions were calculated:

$$\vert Δ X \vert = \frac{1}{n} {\sum }_{i = 1}^{n} \vert {EC}_{i x} - \vert$$

Where |ΔX| represents the mean absolute distance in the x-direction, for a patient having n ECs. ECᵢₓ and [symbol] respectively represent the x-coordinates of the i^{th} EC calculated using the clinical method and FASCILE respectively. Therefore, these values represented FASCILE's localization offset in the 3 directions.

The mean Euclidean distance (D) was also calculated for each patient:

$$D = \frac{1}{n} {\sum }_{i = 1}^{n} \sqrt{{( E {C}_{i x} - )}^{2} + {( E {C}_{i y} - )}^{2} + {( E {C}_{i z} - \hat{E {C}_{lz}} )}^{2}}$$

Note that this was a conservative statistic that exceeded the mean of |ΔX|, |ΔY|, and |ΔZ| for each patient. It represented the largest possible offset in the localization of ECs using FASCILE. The grand mean of Euclidean distances was then calculated across all patients. These calculations were also performed separately for EC's lying in gray and white matter respectively. Using an identical analysis, FASCILE was also compared with Curry 7 (Compumedics, Charlotte, N.C.) which is commonly used for MRI-CT co-registration and electrode segmentation.

### Time Taken for EC Identification, Sorting, and Labeling

FASCILE's convenience was evaluated by comparing the time taken for EC identification, sorting, and labeling by the software developer (BE) and a first-time clinical user (RA). The clinician received one 30-minutes training session on a patient not included in this study. Although the times for clinical EC localization and labeling process were not retained, this time was measured in 5 randomly selected patients, to compare with the speed of FASCILE. The clinical EC identification and labeling was performed by one investigator (LR), both in the routine practice, and for this study.

### Results

Thirty-five consecutive patients (16 females) undergoing SEEG monitoring at Cincinnati Children's Hospital were included, aged 3.6-21.2 years (mean±standard deviation 13.3±4.6). From 7 to 20 electrodes were implanted in these patients (12±3), with 80-253 (133±42) ECs. Brain MRI findings in these patients are described in Table 5.

TABLE 5
Pre-implant brain magnetic resonance imaging in the included patients

| Findings on brain magnetic resonance imaging | # |
|---|---|
| Normal | 4 |
| Normal with evidence of prior surgery | 3 |
| Left temporal tumor resection | |
| Laser ablation | |
| Right frontal operculum resection | |
| Non-specific findings | 7 |
| Non-specific signal change in left inferior temporal gyrus | |
| Non-specific T2 hyper-intensity in left anterior temporal lobe, normal GM-WM | |
| differentiation | |
| Non-specific signal changes in left frontal sub-cortical WM | |
| Non-specific signal changes in left anterior-mesial temporal lobe | |
| Non-specific signal change in right medial temporal lobe, also prior laser ablation | |
| in the same region | |
| Non-specific signal changes in left > right posterior cerebral hemisphere, also | |
| Rathke's cleft cyst | |
| Non-specific signal changes in bilateral sub-cortical WM | |
| Malformations of brain development | 3 |
| Right atrial PV heterotopia, non-specific T2 hyper-intensities in PV WM | |
| Right hemispheric PMG, GM heterotopia, and volume loss | |
| Neurofibromatosis type 1 | |
| Encephalomalacia and/or gliosis | 10 |
| Left frontal encephalomalacia and gliosis | |
| Left occipital and left parietal encephalomalacia | |
| Left hemispheric gliosis | |
| Left peri-atrial gliosis and incomplete inversion of hippocampus | |
| Right hemisphere encephalomalacia and gliosis | |
| Right insular gliosis | |
| Sequelae of prior intra-ventricular hemorrhage, right mesial temporal volume | |
| loss and gliosis, possible right HS, and left frontal ventriculostomy catheter | |
| Bilateral occipital gliosis and encephalomalacia | |
| Bilateral multi-lobar encephalomalacia and small hippocampi | |
| Bilateral peri-ventricular gliosis and callosal thinning | |
| Vascular | 2 |
| Right frontal developmental venous anomaly (incidental) | |
| Multiple cavernous malformations, right frontal surgical cavity | |
| Hippocampal sclerosis + additional pathology | 3 |
| Left HS (n = 2) | |
| Left HS and right > left multi-lobar encephalomalacia | |
| Suspected cortical dysplasia | 3 |
| Left mesial temporal lobe | |
| Left temporal lobe | |
| Right inferior frontal gyrus | |

[GM Gray Matter; HS Hippocampal sclerosis; PMG Polymicrogyria; PV Peri-ventricular; WM White Matter]

### Accuracy

Using FASCILE for identifying, sorting, and labeling ECs from post-operative CT, and comparing with our usual clinical practice of localization with Analyze and manual labeling, we found mean absolute distances of 0.26-0.98, 0.22-0.87, and 0.11-1.33 pixels in the x (lateral), y (antero-posterior), and z (supero-inferior) directions respectively (Table 6). The mean Euclidean distance varied from 0.59 to 1.38 pixels across patients, with a grand mean of 0.91±0.19 pixels. Because the resolution of all co-registered neuroimaging was 0.8 mm/pixel in all directions, the real-world grand mean Euclidean distance for all patients was 0.73±0.15 mm Only 2 patients had a mean Euclidean distance of >1.28 pixels (1.02 mm) exceeding grand mean+2SD. No significant difference was seen for the grand mean distance between EC's in gray (0.74±0.17 mm) and white (0.67±0.19 mm) matter (p=0.977).

TABLE 6
Comparison of FASCILE with manual method using Analyze: localization error
in each direction and mean Euclidean distance

| | Electrodes | Electrode | All EC's | | | | EC's in Gray Matter | | | | EC's in White Matter | | | |
|---|---|---|---|---|---|---|---|---|---|---|---|---|---|---|
| Patient | (n) | Contacts (n) | \|ΔX\| | \|ΔY\| | \|ΔZ\| | Distance | \|ΔX\| | \|ΔY\| | \|ΔZ\| | Distance | \|ΔX\| | \|ΔY\| | \|ΔZ\| | Distance |
| 1 | 7 | 90 | 0.57 | 0.46 | 0.17 | 0.95 | 0.63 | 0.55 | 0.24 | 1.10 | 0.51 | 0.39 | 0.12 | 0.83 |
| 2 | 11 | 124 | 0.35 | 0.42 | 0.31 | 0.87 | 0.28 | 0.50 | 0.35 | 0.91 | 0.29 | 0.31 | 0.24 | 0.71 |
| 3 | 18 | 204 | 0.46 | 0.45 | 0.25 | 0.92 | 0.37 | 0.38 | 0.38 | 0.98 | 0.26 | 0.39 | 0.17 | 0.71 |
| 4 | 12 | 94 | 0.44 | 0.25 | 0.22 | 0.77 | 0.32 | 0.15 | 0.17 | 0.58 | 0.33 | 0.29 | 0.20 | 0.69 |
| 5 | 10 | 102 | 0.58 | 0.72 | 1.33 | 1.03 | 0.48 | 0.38 | 0.35 | 0.91 | 0.61 | 0.28 | 0.28 | 0.96 |
| 6 | 16 | 182 | 0.65 | 0.39 | 0.20 | 1.02 | 0.50 | 0.42 | 0.33 | 0.98 | 0.71 | 0.74 | 0.68 | 1.50 |
| 7 | 11 | 136 | 0.32 | 0.27 | 0.11 | 0.61 | 0.13 | 0.23 | 0.09 | 0.42 | 0.55 | 0.33 | 0.16 | 0.89 |
| 8 | 11 | 118 | 0.80 | 0.29 | 0.20 | 1.10 | 0.81 | 0.25 | 0.23 | 1.10 | 0.74 | 0.28 | 0.18 | 1.05 |
| 9 | 14 | 152 | 0.96 | 0.44 | 0.31 | 1.38 | 0.95 | 0.43 | 0.28 | 1.32 | 0.43 | 0.47 | 0.33 | 1.00 |
| 10 | 14 | 126 | 0.58 | 0.26 | 0.20 | 0.84 | 0.57 | 0.32 | 0.22 | 0.90 | 0.54 | 0.19 | 0.19 | 0.75 |
| 11 | 17 | 232 | 0.52 | 0.80 | 0.29 | 0.98 | 0.43 | 0.57 | 0.45 | 1.08 | 0.39 | 0.70 | 0.58 | 1.25 |
| 12 | 9 | 123 | 0.26 | 0.22 | 0.18 | 0.59 | 0.28 | 0.31 | 0.24 | 0.70 | 0.24 | 0.16 | 0.13 | 0.48 |
| 13 | 9 | 94 | 0.64 | 0.44 | 0.32 | 1.06 | 0.79 | 0.35 | 0.38 | 1.21 | 0.54 | 0.49 | 0.29 | 0.96 |
| 14 | 18 | 208 | 0.27 | 0.31 | 0.30 | 0.71 | 0.31 | 0.32 | 0.35 | 0.80 | 0.22 | 0.31 | 0.25 | 0.63 |
| 15 | 20 | 253 | 0.57 | 0.32 | 0.12 | 0.82 | 0.49 | 0.71 | 0.65 | 1.31 | 0.48 | 0.55 | 0.33 | 1.00 |
| 16 | 11 | 112 | 0.40 | 0.32 | 0.29 | 0.82 | 0.39 | 0.27 | 0.27 | 0.75 | 0.40 | 0.40 | 0.23 | 0.85 |
| 17 | 13 | 120 | 0.35 | 0.55 | 0.64 | 1.19 | 0.39 | 0.48 | 0.70 | 1.22 | 0.31 | 0.62 | 0.58 | 1.17 |
| 18 | 13 | 140 | 0.67 | 0.36 | 0.22 | 1.02 | 0.62 | 0.31 | 0.23 | 0.95 | 0.71 | 0.31 | 0.17 | 1.00 |
| 19 | 9 | 97 | 0.46 | 0.45 | 0.19 | 0.88 | 0.64 | 0.34 | 0.18 | 0.89 | 0.22 | 0.53 | 0.18 | 0.78 |
| 20 | 10 | 80 | 0.34 | 0.26 | 0.20 | 0.66 | 0.41 | 0.27 | 0.25 | 0.75 | 0.28 | 0.19 | 0.11 | 0.52 |
| 21 | 12 | 160 | 0.45 | 0.44 | 0.38 | 0.97 | 0.46 | 0.44 | 0.30 | 0.91 | 0.22 | 0.43 | 0.35 | 0.80 |
| 22 | 12 | 146 | 0.95 | 0.29 | 1.09 | 0.74 | 0.33 | 0.30 | 0.15 | 0.69 | 0.38 | 0.28 | 0.11 | 0.67 |
| 23 | 11 | 89 | 0.37 | 0.58 | 0.33 | 0.96 | 0.38 | 0.62 | 0.36 | 1.00 | 0.27 | 0.50 | 0.17 | 0.74 |
| 24 | 12 | 98 | 0.36 | 0.63 | 1.33 | 1.03 | 0.25 | 0.53 | 0.30 | 0.86 | 0.24 | 0.57 | 0.43 | 0.93 |
| 25 | 10 | 116 | 0.98 | 0.23 | 0.21 | 1.21 | 0.86 | 0.16 | 0.22 | 1.06 | 0.86 | 0.28 | 0.18 | 1.16 |
| 26 | 8 | 90 | 0.28 | 0.39 | 0.26 | 0.78 | 0.36 | 0.38 | 0.29 | 0.83 | 0.15 | 0.39 | 0.22 | 0.68 |
| 27 | 9 | 100 | 0.43 | 0.47 | 0.22 | 0.92 | 0.55 | 0.43 | 0.18 | 0.92 | 0.21 | 0.50 | 0.21 | 0.80 |
| 28 | 12 | 152 | 0.34 | 0.22 | 0.11 | 0.60 | 0.19 | 0.16 | 0.09 | 0.41 | 0.19 | 0.14 | 0.05 | 0.32 |
| 29 | 15 | 150 | 0.74 | 0.45 | 0.34 | 1.14 | 0.50 | 0.43 | 0.33 | 0.93 | 0.33 | 0.32 | 0.28 | 0.74 |
| 30 | 11 | 117 | 0.30 | 0.26 | 0.11 | 0.59 | 0.42 | 0.25 | 0.14 | 0.70 | 0.20 | 0.27 | 0.08 | 0.50 |
| 31 | 11 | 88 | 0.48 | 0.42 | 0.33 | 1.01 | 0.34 | 0.41 | 0.41 | 0.97 | 0.49 | 0.44 | 0.31 | 0.99 |
| 32 | 14 | 164 | 0.73 | 0.87 | 1.02 | 1.18 | 0.82 | 0.43 | 0.17 | 1.22 | 0.64 | 0.54 | 0.12 | 1.10 |
| 33 | 12 | 137 | 0.38 | 0.33 | 0.14 | 0.77 | 0.46 | 0.32 | 0.13 | 0.79 | 0.17 | 0.28 | 0.15 | 0.50 |
| 34 | 13 | 113 | 0.30 | 0.48 | 0.51 | 1.00 | 0.35 | 0.51 | 0.53 | 1.09 | 0.15 | 0.33 | 0.44 | 0.77 |
| 35 | 13 | 154 | 0.50 | 0.49 | 0.22 | 0.94 | 0.46 | 0.54 | 0.24 | 0.97 | 0.42 | 0.43 | 0.13 | 0.80 |
| | | Image | | | | | | | | | | | | |
| | | Distance | | | | | | | | | | | | |
| | | (pixels) | | | | | | | | | | | | |
| | | Mean | 0.51 | 0.41 | 0.36 | 0.92 | 0.47 | 0.38 | 0.29 | 0.92 | 0.39 | 0.39 | 0.25 | 0.83 |
| | | SD | 0.20 | 0.16 | 0.32 | 0.19 | 0.19 | 0.13 | 0.14 | 0.21 | 0.19 | 0.14 | 0.14 | 0.24 |
| | | Real-world | | | | | | | | | | | | |
| | | Distance | | | | | | | | | | | | |
| | | (mm) | | | | | | | | | | | | |
| | | Mean | 0.41 | 0.33 | 0.29 | 0.73 | 0.38 | 0.31 | 0.23 | 0.74 | 0.31 | 0.31 | 0.20 | 0.67 |
| | | SD | 0.16 | 0.13 | 0.26 | 0.15 | 0.15 | 0.10 | 0.11 | 0.17 | 0.15 | 0.12 | 0.12 | 0.19 |

[FASCILE Fast Automated Stereo-EEG Electrode Contact Identification and Labeling Ensemble, SD Standard Deviation. Note that the distances for individual patients are in pixel dimensions].

Compared to Curry 7, the mean Euclidean distances were 0.33 to 1.55 pixels across all patients, with the grand mean being 0.82±0.18 pixels, which equates to 0.41±0.09 mm (Table 7). Again, the localization offset was not significantly different between EC's in gray (0.42±0.08 mm) and white (0.42±0.09 mm) matter (p=0.999).

TABLE 7
Comparison of FASCILE with Curry: localization error in each direction and
mean Euclidean distance

| | Electrodes | Electrode | All EC's | | | | EC's in Gray Matter | | | | EC's in White Matter | | | |
|---|---|---|---|---|---|---|---|---|---|---|---|---|---|---|
| Patient | (n) | Contacts (n) | \|ΔX\| | \|ΔY\| | \|ΔZ\| | Distance | \|ΔX\| | \|ΔY\| | \|ΔZ\| | Distance | \|ΔX\| | \|ΔY\| | \|ΔZ\| | Distance |
| 1 | 7 | 90 | 0.33 | 0.33 | 0.33 | 0.33 | 0.36 | 0.32 | 0.41 | 0.73 | 0.10 | 0.28 | 0.33 | 0.72 |
| 2 | 11 | 124 | 0.30 | 0.38 | 0.43 | 0.72 | 0.28 | 0.37 | 0.46 | 0.74 | 0.13 | 0.29 | 0.36 | 0.72 |
| 3 | 18 | 204 | 0.34 | 0.33 | 0.51 | 0.80 | 0.36 | 0.31 | 0.51 | 0.78 | 0.33 | 0.33 | 0.48 | 0.78 |
| 4 | 12 | 94 | 0.30 | 0.43 | 0.45 | 0.79 | 0.30 | 0.38 | 0.44 | 0.75 | 0.31 | 0.44 | 0.50 | 0.82 |
| 5 | 10 | 102 | 0.30 | 0.40 | 0.58 | 0.86 | 0.34 | 0.40 | 0.56 | 0.86 | 0.29 | 0.38 | 0.58 | 0.84 |
| 6 | 16 | 182 | 0.32 | 0.35 | 0.48 | 0.77 | 0.31 | 0.36 | 0.51 | 0.79 | 0.38 | 0.32 | 0.51 | 0.81 |
| 7 | 11 | 136 | 0.31 | 0.33 | 0.52 | 0.78 | 0.34 | 0.32 | 0.52 | 0.80 | 0.25 | 0.35 | 0.53 | 0.76 |
| 8 | 11 | 118 | 0.32 | 0.32 | 0.51 | 0.79 | 0.31 | 0.29 | 0.52 | 0.78 | 0.32 | 0.36 | 0.52 | 0.82 |
| 9 | 14 | 152 | 0.31 | 0.36 | 0.54 | 0.81 | 0.31 | 0.38 | 0.54 | 0.81 | 0.31 | 0.35 | 0.56 | 0.81 |
| 10 | 14 | 126 | 0.33 | 0.33 | 0.49 | 0.77 | 0.34 | 0.32 | 0.51 | 0.80 | 0.33 | 0.35 | 0.47 | 0.76 |
| 11 | 17 | 232 | 0.33 | 0.42 | 0.55 | 0.86 | 0.30 | 0.40 | 0.55 | 0.83 | 0.36 | 0.42 | 0.55 | 0.88 |
| 12 | 9 | 123 | 0.32 | 0.37 | 0.50 | 0.79 | 0.30 | 0.34 | 0.49 | 0.75 | 0.34 | 0.40 | 0.50 | 0.83 |
| 13 | 9 | 94 | 0.45 | 0.43 | 0.68 | 1.05 | 0.56 | 0.39 | 0.76 | 1.17 | 0.39 | 0.45 | 0.63 | 0.97 |
| 14 | 18 | 208 | 0.38 | 0.35 | 0.58 | 0.88 | 0.41 | 0.33 | 0.52 | 0.85 | 0.33 | 0.35 | 0.63 | 0.90 |
| 15 | 20 | 253 | 0.31 | 0.38 | 0.51 | 0.82 | 0.32 | 0.37 | 0.48 | 0.78 | 0.31 | 0.40 | 0.56 | 0.86 |
| 16 | 11 | 112 | 0.26 | 0.29 | 0.39 | 0.63 | 0.27 | 0.26 | 0.41 | 0.63 | 0.25 | 0.31 | 0.37 | 0.64 |
| 17 | 13 | 120 | 0.28 | 0.33 | 0.51 | 0.74 | 0.31 | 0.35 | 0.58 | 0.81 | 0.26 | 0.31 | 0.46 | 0.69 |
| 18 | 13 | 140 | 0.30 | 0.37 | 0.58 | 0.85 | 0.29 | 0.33 | 0.57 | 0.81 | 0.34 | 0.40 | 0.60 | 0.90 |
| 19 | 9 | 97 | 0.30 | 0.38 | 0.58 | 0.85 | 0.34 | 0.35 | 0.55 | 0.83 | 0.26 | 0.41 | 0.59 | 0.84 |
| 20 | 10 | 80 | 0.27 | 0.34 | 0.45 | 0.71 | 0.24 | 0.36 | 0.45 | 0.71 | 0.30 | 0.33 | 0.43 | 0.70 |
| 21 | 12 | 160 | 0.32 | 0.46 | 0.51 | 0.85 | 0.31 | 0.48 | 0.48 | 0.85 | 0.33 | 0.43 | 0.53 | 0.83 |
| 22 | 12 | 146 | 0.32 | 0.33 | 0.40 | 0.70 | 0.30 | 0.33 | 0.38 | 0.68 | 0.32 | 0.34 | 0.44 | 0.71 |
| 23 | 11 | 89 | 0.28 | 0.36 | 0.42 | 0.72 | 0.28 | 0.31 | 0.41 | 0.69 | 0.27 | 0.37 | 0.43 | 0.72 |
| 24 | 12 | 98 | 0.28 | 0.43 | 0.45 | 0.79 | 0.26 | 0.42 | 0.48 | 0.79 | 0.32 | 0.45 | 0.43 | 0.82 |
| 25 | 10 | 116 | 0.34 | 0.43 | 0.51 | 0.84 | 0.34 | 0.42 | 0.49 | 0.82 | 0.30 | 0.35 | 0.49 | 0.76 |
| 26 | 8 | 90 | 0.30 | 0.36 | 0.47 | 0.77 | 0.24 | 0.40 | 0.46 | 0.76 | 0.36 | 0.34 | 0.48 | 0.79 |
| 27 | 9 | 100 | 0.27 | 0.37 | 0.46 | 0.75 | 0.30 | 0.35 | 0.44 | 0.73 | 0.25 | 0.39 | 0.48 | 0.76 |
| 28 | 12 | 152 | 0.27 | 0.48 | 0.48 | 0.82 | 0.31 | 0.45 | 0.46 | 0.82 | 0.23 | 0.51 | 0.49 | 0.83 |
| 29 | 15 | 150 | 0.34 | 0.41 | 0.58 | 0.89 | 0.36 | 0.34 | 0.61 | 0.88 | 0.31 | 0.42 | 0.51 | 0.85 |
| 30 | 11 | 117 | 0.35 | 1.18 | 0.56 | 1.55 | 0.40 | 0.94 | 0.51 | 1.31 | 0.30 | 1.43 | 0.62 | 1.80 |
| 31 | 11 | 88 | 0.30 | 0.32 | 0.43 | 0.72 | 0.31 | 0.31 | 0.40 | 0.71 | 0.30 | 0.34 | 0.44 | 0.71 |
| 32 | 14 | 164 | 0.75 | 0.29 | 0.48 | 1.01 | 0.80 | 0.31 | 0.46 | 1.05 | 0.76 | 0.25 | 0.50 | 1.01 |
| 33 | 12 | 137 | 0.33 | 0.36 | 0.52 | 0.81 | 0.32 | 0.37 | 0.49 | 0.78 | 0.31 | 0.30 | 0.48 | 0.75 |
| 34 | 13 | 113 | 0.42 | 0.72 | 0.71 | 1.23 | 0.47 | 0.91 | 0.81 | 1.46 | 0.33 | 0.49 | 0.64 | 0.97 |
| 35 | 13 | 154 | 0.33 | 0.39 | 0.48 | 0.80 | 0.31 | 0.39 | 0.49 | 0.79 | 0.31 | 0.40 | 0.49 | 0.80 |
| | | Image | | | | | | | | | | | | |
| | | Distance | | | | | | | | | | | | |
| | | (pixels) | | | | | | | | | | | | |
| | | Mean | 0.33 | 0.40 | 0.50 | 0.82 | 0.34 | 0.39 | 0.51 | 0.83 | 0.31 | 0.40 | 0.50 | 0.83 |
| | | SD | 0.08 | 0.15 | 0.07 | 0.18 | 0.10 | 0.14 | 0.09 | 0.17 | 0.10 | 0.19 | 0.07 | 0.18 |
| | | Real-world | | | | | | | | | | | | |
| | | Distance | | | | | | | | | | | | |
| | | (mm) | | | | | | | | | | | | |
| | | Mean | 0.17 | 0.20 | 0.25 | 0.41 | 0.17 | 0.19 | 0.25 | 0.42 | 0.16 | 0.20 | 0.25 | 0.42 |
| | | SD | 0.04 | 0.08 | 0.04 | 0.09 | 0.05 | 0.07 | 0.04 | 0.08 | 0.05 | 0.09 | 0.04 | 0.09 |

[FASCLE Fast Automated Stereo-EEG Electrode Contact Identification and Labeling Ensemble, SD Staridard Deviation. Note that the distances for individual patients are in pixel dimensions].

### Ease-of-Use and Speed

The time taken for sorting and labeling ECs with FASCILE varied from 7.0-12.0 minutes (mean±SD 10.0±1.9) for the developer (BE) and from 6.5-21.0 minutes (10.7±5.5) for the clinical user (RA), with no significant difference (standardized mean difference [Hedge's ‘g’]±standard error −0.17±0.24 minutes, p=0.481).

In 5 randomly selected patients having 108-208 ECs, the clinical process of sorting and labeling ECs required 145.2-255.8 minutes (198.3±42.5), which was significantly longer than that taken with FASCILE both for the developer (13.3±1.6, p<0.001) and the clinical user (12.5±1.5, p<0.001). In the same 5 patients, the time taken for segmentation of EC's and labeling using Curry was 35.2±13.2 minutes.

This study demonstrated the accuracy, speed, and convenience of FASCILE, a software for identification, sorting, and labeling of SEEG ECs, in 35 consecutive DRE patients. FASCILE is versatile in that it can take different types of input including CT, MRI, or CT-MRI co-registered using a variety of platforms such as Analyze and SPM12. Compared to the clinical method, FASCILE differed in its coordinate locations, overall, by less than one mm, while being over 12-times faster. It has a convenient user interface and can be learned promptly, as reflected by nearly identical processing times for the software developer and a first-time clinical user. It has different output formats useful for clinical practice, research, and neuro-navigation. FASCILE will be particularly useful to centers using SEEG that do not have dedicated personnel for image processing.

### Clinical Utility

Epilepsy teams usually want to visualize the locations of ECs soon after the implantation of SEEG electrodes, to understand the neuroanatomy of seizure generation and propagation. Since the SEEG reading montage is created arbitrarily, ECs lying on different electrodes, which may be anatomically proximate, can sometimes end up being apart on the reading montage, which can potentially affect proper understanding of the seizure propagation. Also, 3D visualization of SEEG ECs is important to synthesize information from ictal recordings and functional brain mapping for surgical planning.

Although there are multiple independent software and toolboxes in programming languages for MRI and CT co-registration (49), there is not an easy solution for assigning SEEG ECs to respective electrodes, labeling, and visualization. At the center, this was performed manually, which required sustained concentration and repetitive mouse movements over 2-4 hours. To compare, it took approximately 10 minutes, even for an inexperienced user, to accomplish identification, sorting, and labeling of SEEG ECs using FASCILE. Also, there was no loss of accuracy, as shown by the mean Euclidean distance of 0.73±0.15 mm across 35 patients, including those with aberrant anatomy (Tables 5 and 6). Compared to Curry, a software often used for imaging co-registration and segmentation of EC's, FASCILE was again accurate with a mean Euclidean distance of 0.41±0.09 mm and nearly 3-times faster (Table 7). The localization error of FASCILE was actually finer than the resolution of the images used (0.8³ mm³/voxel) and did not affect surgical decisions which are based on individual anatomy of sulci and gyri.

FASCILE also generated an NIfTI overlay file containing locations of ECs, which could be integrated with structural or functional neuroimaging data from other modalities in a neuro-navigational system for intra-operative guidance to the neurosurgeon.

Group-Level Analysis with FASCILE

FASCILE was not constrained to co-registered images in individual patient brain space. It could be used to localize ECs in standard brain space for group-level analyses, or for comparisons across patients, as shown previously (60, 61). FASCILE also allowed projection of the ECs onto standard atlases, without warping the atlases into the patient space. However, EC coordinates found in patient space cannot easily be transformed to standard brain space, because modern warping algorithms are nonlinear, having hundreds or thousands of degrees of freedom (62).

### Computational Methods for SEEG Visualization

In 4 DRE patients, an automated method for localization of ECs, by convolving an approximate EC pattern over skull-stripped CT, has been described (63). However, localization error was not reported, and labeling was probably done post-hoc. A study adapted open-source software, 3D Slicer and Freesurfer, for use with SEEG, but the process required 6-24 hours, compared to their conventional process which took 36 hours to 3 days for sorting and labeling ECs (64). The authors validated their methodology in 4 patients having a total of 25 depth electrodes. Although they did not report localization error, they found good inter-user agreement (κ=0.88) (64). Another study using 3D Slicer in 4 patients found mean Euclidean distance varying from 0.4-7.8 mm across electrodes (65). In 3 of the 4 patients, at least one electrode had an error of >3 mm A MATLAB toolbox for co-registration and visualization of SEEG has been described (called iELVis), allowing only manual identification of electrodes (49). While iELVis allowed interactive 3D visualization, it required 30-60 minutes of user time, and 12-24 hours of computer time to localize and visualize electrodes in one brain (49).

A faster pipeline using Medical Imaging Interaction Toolkit reported a mean error of 0.7 mm at the tip of the electrode in 224 electrodes (66). However, this automated approach excluded 29 ECs (1.6%), and its accuracy deteriorated with first-time users. In 12 patients with 200 ECs, a custom algorithm called DEETO, based on Insight Toolkit, was reported to have low error (0.5±0.06 mm) compared to manual segmentation (67). This software also output triangular meshes in estimated electrode positions as a vtk polydata file. Although authors reported a processing time of <1 s, it may be potentially deceptive because DEETO requires significant pre-processing. It relies on planned entry and target points and can miss ECs if the electrode deviates from the expected axis. In comparison, FASCILE offered a free, convenient, fast, and accurate solution for identifying, sorting, and labeling ECs, with different output formats that could be integrated with other imaging data, used for clinical discussions, and research.

### REFERENCES

1. Arya R, et al., Epilepsy Behav. 2019a;99:106455.
2. Arya R, and Crone N E. Epilepsy Behav. 2018; 82:200-1.
3. Arya R, et al., Epilepsy Behav. 2019b;99:106395.
4. Arya R, et al., Epilepsy Behav. 2018a;79:26-33.
5. Arya R, et al., Epilepsia 2013; 54(5):828-39.
6. Arya R, et al., Epilepsy Res. 2019c;151:17-23.
7. Arya R, et al., Epilepsia 2017; 58(4):663-73.
8. Arya R, et al., Epilepsia 2018b;59(4):792-801.
9. Aungaroon G, et al., Clin Neurophysiol. 2017; 128(10):2078-86.
10. Austermuehle A, et al., Ann Neurol. 2017; 81(4):526-37.
11. Babajani-Feremi A, et al., Clin Neurophysiol. 2016; 127(3):1822-36.
12. Bauer P R, et al., Brain Stim. 2013; 6(4):524-31.
13. Chang E F, et al., J Neurosurg. 2015; 122(2):250-61.
14. Cuisenier P, et al., J Neurosurg. 2020:1-11.
15. Giussani C, et al., Neurosurgery 2010; 66(1):113-20.
16. Goldmann R E, et al., Epilepsy Behav. 2005; 6(4):473-87.
17. Gramfort A, et al., Front Neurosc. 2013; 7:267.
18. Hamberger M J., Neuropsychol Rev. 2007; 17(4):477-89.
19. Jayakar P. et al., Epilepsia 2014; 55(4):507-18.
20. Koessler L, et al., Hum Brain Mapp. 2017; 38(2):974-86.
21. Kojima K, et al., Clin Neurophysiol. 2013; 124(5):857-69.
22. Kombos T, and Suss O., Neurosurg Focus 2009; 27(4):E3.
23. Kuchcinski G, et al., Neurology 2015; 84(6):560-8.
24. Kursa M B, and Rudnicki W R., J. Stat Softw 2010; 36(11):1-13.
25. Liegeois F, et al., Neuropsychologia 2008; 46(13):3101-7.
26. Maris E, et al., J. Neurosc Methods 2007; 164(1):177-90.
27. Miller K J, et al., J. Neurosurg Pediatr. 2011; 7(5):482-90.
28. Mooij A H, et al., Clin Neurophysiol. 2016; 127(2):1113-9.
29. Mullin J P, et al., Epilepsia 2016; 57(3):386-401.
30. Nakai Y, et al., Brain 2017; 140(5):1351-70.
31. Ogawa H, et al., World Neurosurg. 2017; 97:123-31.
32. Price C T., J. Anat. 2000;197 Pt 3:335-59.
33. Price C T. Ann N Y Acad Sc. 2010; 1191:62-88.
34. R Development Core Team. R: A Language and Environment for Statistical Computing Vienna, Austria: R Foundation for Statistical Computing 2019.
35. Rattay F., Neuroscience 1999; 89(2):335-46.
36. Ruescher J et al., Neurolmage 2013; 81:164-77.
37. Sakpichaisakul K, et al., Seizure 2020; 80:183-91.
38. Sinai A, et al., Brain 2005;128(Pt 7):1556-70.
39. Tamura Y, et al., J. Neurosurg. 2016; 125(6):1580-8.
40. Towle V L, et al., Brain 2008;131(Pt 8):2013-27.
41. Trebuchon A, et al., J. Clin Neurophysiol. 2016; 33(6):511-21.
42. Wang Y, et al., Neurology 2016; 86(13):1181-9.
43. Yarkoni T, et al., Nat Methods 2011; 8(8):665-70.
44. Young J J, et al., Epilepsy Behav. 2018; 84:148-51.
45. Zea Vera A, et al., Clin Neurophysiol. 2017; 128(10):2087-93.
46 Jayakar P, et al., Epilepsia. 2016 November; 57(11):1735-47.
47 Abou-Al-Shaar H, et al., J. Clin. Neurosci. 2018; 53:132-34.
48 Robb R A, et al., Computerized Medical Imaging and Graphics. 1989; 13(6):433-54.
49 Groppe D M, J. Neurosci. Methods. 2017; 281:40-48.
50 Arya R, et al., J. Neurosurgery Pediatrics. 2019 May 31:1-9.
51 Brett M, et al., Freec84.(2019). nipy/nibabel: 3.0. 0 (3.0. 0)[Computer software]. Zenodo.
52 Lundh F. An introduction to tkinter. Pythonware website 1999.
53 Hunter J D. Computing in Science & Engineering. 2007; 9(3):90-95.
54 Van Rossum G, and Drake F. Python 3 Reference Manual (Create-Space). Scotts Valley: CA; 2009.
55 Walt Svd, et al., Computing in Science & Engineering. 2011; 13(2):22-30.
56 Van der Walt S, et al., PeerJ. 2014;2:e453.
57 Canny S. python-pptx Documentation. 2019.
58 Virtanen P, et al., J. Nuclear Med. Tech. 2007; 35(3):115-28.
59 Isnard J et al., Neurophysiologie clinique 2018 February; 48(1):5-13.
60 Arya R, et al., Clin. Neurophysiol. 2020 August; 131(8):1691-701.
61 Ervin B, et al., Clin. Neurophysiol. 2020.
62 Cohen D S, et al., J. Neurosurgery. 1995; 82(5):772-79.
63 Hofmanis J, et al., BIOSIGNALS2011. p. 459-62.
64 Princich J P, et al., Frontiers in Neuroscience. 2013; 7:260.
65 Benadi S, et al., Healthc Technol Lett. 2018 Oct.; 5(5):215-20.
66 Granados A, et al., Intl. J. Computer Assisted Radiology And Surgery. 2018 Jun.; 13(6):935-46.
67 Arnulfo G, et al., BMC bioinformatics. 2015 Mar. 25; 16:99.
68 Mehrtash A, et al., IEEE transactions on medical imaging. 2018; 38(4):1026-36.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A method for mapping a functional brain site in a subject, comprising:
a) subjecting a subject to a task;
b) performing an intracranial electroencephalography (iEEG) assay to the subject while the subject is performing the task, wherein the iEEG assay comprises multiple stereotactic or subdural electrodes placed at multiple sites within the subject's brain;
c) recording high gamma EEG signals during step (b) at each iEEG electrode;
d) analyzing the high gamma EEG signals to produce a collection of output data parameters; and e) identifying a functional brain site in the subject that is responsible for performing the task based the collection of output data parameters determined in step (d).

2. The method of claim 1, wherein the subject is a human patient.

3. The method of claim 1, wherein the subject is a human child.

4. The method of claim 1, wherein the subject is a human adult.

5. The method of claim 1, wherein the subject has a brain disorder.

6. The method of claim 5, the brain disorder is selected from the group consisting of epilepsy, a brain tumor, and a vascular lesion.

7. The method of claim 6, wherein the subject is a human patient with drug-resistant epilepsy (DRE).

8. The method of claim 1, wherein the method is performed to the subject prior to a brain surgery.

9. The method of claim 1, wherein the high gamma iEEG signals comprise signals at about 50-150 Hz.

10. The method of claim 9, the method further comprises determining a site for the brain surgery based on the functional brain site identified in step (e).

11. The method of claim 1, wherein the analyzing step (d) comprises calculating time-frequency representations (TFRs) for frequency bands ranging from about 50-150 Hz, optionally with 1 Hz step.

12. The method of claim 1, wherein the analyzing step (d) comprises a clustering algorithm.

13. The method of claim 12, wherein the clustering algorithm is a Maris-Oostenveld nonparametric permutation-based clustering procedure.

14. The method of claim 1, wherein the analyzing step (d) comprises locating the positions of the electrode contacts.

15. The method of claim 14, wherein the positions of the electrode contacts are determined by manual process or by computer-aided process.

16. The method of claim 15, wherein the computer-aided process comprise the utilization of FASCILE.

17. The method of claim 1, wherein the functional brain site is a language site and the task is visual naming, auditory naming, story listening, or conversational speech.

18. The method of claim 1, wherein the functional brain site is a motor site and the task is visually-cued hand motor task.

19. The method of claim 1, wherein the method further comprises comparing the mapping results obtained in step (e) with a reference database.

20. The method of claim 1, wherein the method further comprises performing ESM to the subject and comparing the mapping results obtained in step (e) with the results from the ESM.
